(19) United States
(12) Patent Application Publication
(10) Pub. No.: US 2016/0235703 A1
(43) Pub. Date: Aug. 18, 2016

(54) Novel Uses of Neuraminidase Inhibitors in Infectious Diseases

(71) Applicant: Jack J. Broadhurst, Pinehurst, NC (US)

(72) Inventor: Jack J. Broadhurst, Pinehurst, NC (US)

(21) Appl. No.: 15/138,144

(22) Filed: Apr. 25, 2016

Related U.S. Application Data
(62) Division of application No. 14/169,076, filed on Jan. 30, 2014.
Application No. 14/169,076 is a continuation-in-part of application No. 13/612,739, filed on Sep. 12, 2012, now abandoned.
Application No. 13/612,739 is a continuation-in-part of application No. 13/024,285, filed on Feb. 9, 2011, now abandoned.
Application No. 13/024,285 is a continuation of application No. 11/112,138, filed on Apr. 22, 2005, now Pat. No. 7,910,620.

(51) Int. Cl. A61K 31/215

(52) U.S. Cl. CPC A61K 31/215

(57) ABSTRACT

The present invention relates to methods of decreasing the infectivity, morbidity and rate of mortality, in treating diseases associated with a variety of pathogenic organisms, specifically diseases involving one or more pathogens that require neuraminidase as a virulence factor. In addition, the present invention uses biology based therapy to treat neuraminidase dependent infections or diseases dependent on sialic acid metabolism.

### CROSS-REFERENCE TO RELATED APPLICATIONS

This application is a divisional of commonly owned U.S. patent application Ser. No. 14/169,076 filed on Jan. 30, 2014, which is a continuation-in-part of commonly owned U.S. patent application Ser. No. 13/612,739 filed on Sep. 12, 2012, which is a continuation-in-part of U.S. patent application Ser. No. 13/024,285 filed on Feb. 9, 2011, which is a continuation of U.S. patent application Ser. No. 11/112,138, now U.S. Pat. No. 7,910,620, filed on Apr. 22, 2005. The entire contents of these prior patent applications are hereby incorporated by reference.

### BACKGROUND

Many disease causing microorganisms, such as bacteria, fungi, and viruses, play a significant role in producing a myriad of diseases and conditions in humans and animals. Due to their widespread capability of pathogenic infectivity, morbidity and mortality, considerable activity has been devoted towards developing convenient effective methods to help prevent or treat these diseases caused by these pathogens.

For example, viruses such as influenza, have a high mortality rate in humans and are devastating to man and animals. It is estimated that more than $1 billion per year is lost in productivity from absence due to sickness from an influenza virus infection.

With respect to clinical veterinary medicine, there are many diseases, viral, bacterial, fungal and protozoan, that are detrimental to animals. Viruses, bacteria, protozoan and fungi that cause diseases that effect animals in the food industry, for example, cattle, pigs and chickens can be quite costly and result in billions of dollars lost in the food industry. These same microorganisms can wipe out large masses of domestic animals, such as cats and dogs, since they can be highly contagious and spread quickly, thus being detrimental to veterinary hospitals, kennels, and breeding facilities, resulting in both emotional and monetary loss. Recently, there have been several disease causing microorganisms that have jumped the species barrier, resulting in new variant diseases that are fatal to man.

Canine parvovirus (CPV), for example, has a high morbidity and mortality rate and is a life threatening infection that has been estimate to affect up to 1 million dogs per year in the United States. The disease resulting from parvovirus is typically almost always fatal, and there have been very few major advances in the way that dogs with canine parvovirus are treated. As a result, the disease is typically associated with a significant mortality rate. Most of the untreated dogs succumb to the diseases, and even with care, for example, in private practice, mortality rate still is quite high. In addition, the disease from a parvovirus infection is costly, both monetarily and emotionally for the dog's caretakers.

With canine parvovirus, the clinical disease is often characterized by fever, acute gastroenteritis, which can progress rapidly to shock and death. Septicemia and endotoxemia can play an important role in the pathogenesis of canine parvovirus. It has been found that when gnotobiotic (germ free) dogs were infected with canine parvovirus, they did not develop any signs of the illness. Similar findings were made with germ-free cats when exposed with highly pathogenic feline parvovirus. Thus, attempts have been directed to utilize treatments aimed at preventing or treating septicemia and endotoxemia. Unfortunately, these treatments have shown little or no benefit on survival of these animals.

Necrotic enteritis (NE), caused by Clostridium perfringens, is an infectious disease that has been found to have a major impact on poultry production globally, with estimated annual economic loss in the billions. Currently, the use of antibiotics has resulted in antibiotic resistance strains.

Similarly, diseases caused by fungal (including yeast) and protozoan infections, can be also be difficult and costly to treat. For example, fungal infections such as those caused by Candida species (spp) can be a complicated series of antibiotic, steroid and immunosuppressive therapies, that may show little or no benefit to animals infected by these diseases. Typically, antifungal treatments can take months of various antifungal drugs, each with potential harm to the animal's organs.

Typically, Candida infections are reported in areas that contain a layer of mucin between the epithelial cells of the organ and lumen of the organ system. Historically, Candida infections can be difficult to treat requiring months of therapy using different antifungal drugs. Antifungal drugs are known to be toxic to the host's liver and kidney, and so any therapy that shortens the treatment period is significant. Additionally, the use of antibiotics has been known to those skilled in the art to promote and enhance growth of yeast.

Conventional methods towards the control of these disease causing microorganisms or pathogens, include vaccination, drug therapy and public health measures. Typically, one method of treatment of these types of diseases is antibiotic therapy, which has been found to be effective against diseases caused by bacteria. Although an invaluable advance, there are disadvantages of using antibiotic therapy, especially when strains of bacteria appear to be resistant to antibiotics.

Vaccines have also been used to treat diseases caused by viruses. However, there can be disadvantages involved with the production of suitable vaccines. First, the vaccines derived from whole killed or whole attenuated viruses, may retain residual disease causing activity. Further, vaccines typically are reformulated each year in response to antigenic variation and are known to be ineffective against new viral variants.

Additional disadvantages are that medications typically can be expensive, especially if animals are on antibiotics, for example, over a long course of time, eventually often resulting in an agonizing imminent death of these animals.

As those skilled in the art would appreciate, there is a need for methods that can decrease the infectivity, morbidity and mortality associated with exposures to such pathogens. Such compositions and methods of treatment should preferably not have the undesirable properties of promoting microbial resistance, or being toxic to the recipient. Still further, there is a need for treatment and prevention in diseases caused by microorganisms that are cost effective and do not take a long period of time. In addition, there is a need to provide treatment of infectious diseases by developing biology based therapies.

### SUMMARY

The present invention is directed towards a method and treatment that meets these needs.

This invention provides a method of inhibiting, treating and preventing mucosal diseases, diseases associated with neuraminidase dependent bacteria, fungal, yeast, protozoan and superinfections with a neuraminidase inhibitor.

In a preferred embodiment, the present invention uses biology based therapy to treat infectious diseases that have been previously treated with antibiotics, antivirals, or anti-fungals, alone or in combination, with limited success. Where there has been variable success in viruses with antiviral drugs, and antibiotics (conventional therapy), or anti-fungal drugs, neuraminidase inhibitors according to the present invention have been proven to be successful and predictable. In a most preferred embodiment of the present invention, when neuraminidase inhibitors are used in these same diseases, the results have been dramatic.

Further, this invention relates to a means for reducing the severity of or preventing a neuraminidase dependent bacterial infection of the mucousal membrane tract following a viral infection by administering an effective amount of a neuraminidase inhibitor alone or in combination with a pharmaceutically acceptable compound prior to or during the course of the neuraminidase dependent bacterial infection, during the course of the superinfection or during the course of the coinfection.

Still further, this invention relates to a means for reducing the severity of or preventing a neuraminidase dependent fungal, yeast and protozoan infection by administering an effective amount of a neuraminidase inhibitor alone or in combination with a pharmaceutically acceptable compound prior to or during the course of the neuraminidase dependent infection, during the course of a superinfection or during the course of a coinfection.

In one embodiment, the present invention provides methods used for preventing disease or treating animals, including humans, exposed to pathogens or the threat of pathogens.

In still a further embodiment of the present invention, there is a method used for preventing animals, including humans, from getting a disease associated with the specific pathogen. For example, the animal is contacted with effective amounts of the compositions prior to exposure to pathogenic organisms. In other embodiments, the animal is contacted with effective amounts of the composition after exposure to pathogenic organisms. Thus, the present invention provides a method of both prevention and treatment of microbial, fungal, yeast and protozoan infections.

In preferred embodiments, the present invention provides methods to decrease pathogenic organism infectivity, morbidity and mortality, by using an effective method of treatment where the composition comprises a compound that can include neuraminidase inhibitors.

In some preferred embodiment, the compound comprising a neuraminidase inhibitor is oseltamivir (TAMIFLU®, hereinafter referred to as TAMIFLU).

In another aspect of the present invention, the composition can include additional compounds, such as antibiotics, and/or antifungal drugs, for example, which can be used in addition to the compound comprising the neuraminidase inhibitor.

In specific embodiments of the present invention, the method or treatment is performed for a sufficient amount of time to reduce the virulence factor of the pathogen.

In yet another specific embodiment, the pathogen can be selected from the group consisting of bacteria, fungi, yeast and protozoan.

In a most preferred embodiment, the current invention provides a method of using neuraminidase inhibitors to treat: 1) infections involving neuraminidase dependent bacteria other than mucosal surfaces (blackleg, necrotic dermatitis), 2) one or more bacteria involving mucosal surfaces (colibacillosis or enteriopathic E. coli in all species, respiratory, renal, uterine, and mammary gland infections involving neuraminidase producing bacteria, Salmonellosis in all species, Bordetella and Pasteurella respiratory infection in all species) and 3) superinfections that do involve mucosal surfaces (gastrointestinal, respiratory in all species).

In yet another preferred embodiment, the present invention provides a method of using an antiviral drug patented for human influenza to treat neuraminidase dependent bacterial infections, superinfections and coinfections which do not involve the human influenza virus A and/or B, for example, in clinical veterinary medicine.

In still another preferred embodiment, the present invention provides unexpected results of almost 100% effectiveness when used at 1 mg/lb every 12 hours for 10 treatments for therapeutic use and every 24 hours for 5 treatments for prophylactic use.

Finally, the present invention provides the use of a neuraminidase inhibitor to treat diseases involving neuraminidase dependent bacteria.

In a preferred embodiment the use of a neuraminidase inhibitor has been used to inhibit, treat and prevent neuraminidase dependent bacterial infections that are not viral generated dependent infections, from a disease causing microorganism dependent on sialic acid metabolism, comprising administering to an animal in need thereof a therapeutically effective amount of composition comprising one or more compounds, wherein one of the compounds comprises a neuraminidase inhibitor, wherein the neuraminidase inhibitor is any compound that is a structural homologue of sialic acid.

More preferably, structural homologue can defined as any compound that is corresponding in structure and origin but not necessary in function of sialic acid.

In a preferred embodiment, the neuraminidase dependent bacterial infection, is selected from the group consisting of Enterococcus faecalis, Clostridium perfringens and antibiotic resistant bacteria.

Preferably, the animal can be cold blooded or warm blooded. Animal includes but is not limited to human beings, canine, feline, bovine, equine, avian, porcine and any other species known to those skilled in the art, for example, sheep goats and rabbits.

In a preferred embodiment of the present invention, the use of a neuraminidase inhibitor has been used to treat necrotic enteritis (NE) in poultry. More preferably, the neuraminidase inhibitor comprises Tamerindus Indicus and Combretum fragrans.

In yet another preferred embodiment of the present invention, the use of a neuraminidase inhibitor has been used to treat feline Enterobacteria faecalis sinusitis. More preferably, the neuraminidase inhibitor is oseltamivir (TAMIFLU®).

In the most preferred embodiment of the present invention, oseltamivir (TAMIFLU®) has been used to treat canine and feline parvoviral enteritis, canine kennel cough, feline upper respiratory infections, feline nephritis secondary to E. coli, parvoviral enteritis in raccoons and feline Enterobacteria faecalis sinusitis. Given the unique and universal role that sialic acid is known to play in infectious diseases involving neuraminidase dependent bacteria, the concept in the use of a neuraminidase inhibitor would be successful in treating all diseases involving these bacteria regardless of animal species is expected. Animal includes but is not limited to human beings, canine, feline, bovine, equine, avian, porcine and any other species known to those skilled in the art, for example, sheep goats and rabbits.

In yet another most preferred embodiment, the current invention provides a method of using neuraminidase inhibitors to treat, prevent and inhibit infections involving neuraminidase dependent pathogens, wherein the pathogen is capable of producing neuraminidase as a virulence factor,

Several fungal species are known to have neuraminidase activity including Candida fumata.

Several protozoan species are known to have neuraminidase activity including but not limited to tritrichomonase foetus.

In yet another preferred embodiment, the present invention provides a method of using an antiviral drug patented for human influenza to treat neuraminidase dependent bacterial infections, superinfections and coinfections which do not involve the human influenza virus A and/or B, for example, in clinical veterinary medicine.

In still another preferred embodiment, the present invention provides unexpected results of almost 100% effectiveness when used at 1 mg/lb every 12 hours for 10 treatments for therapeutic use and every 24 hours for 5 treatments for prophylactic use.

Finally, the present invention provides the use of a neuraminidase inhibitor to treat diseases involving neuraminidase dependent bacteria.

In the most preferred embodiment of the present invention, oseltamivir (TAMIFLU®) has been used to treat canine and feline parvoviral enteritis, canine kennel cough, feline upper respiratory infections, feline nephritis secondary to E. coli, and parvoviral enteritis in raccoons. Given the unique and universal role that sialic acid is known to play in infectious diseases involving neuraminidase dependent bacteria, the concept in the use of a neuraminidase inhibitor would be successful in treating all diseases involving these bacteria regardless of animal species is expected. Animal includes but is not limited to human beings, canine, feline, bovine, equine, avian, porcine and any other species known to those skilled in the art, for example, sheep goats and rabbits.

In yet another preferred embodiment of the invention, there is provided a method for inhibiting, treating and preventing, neuraminidase dependent infections from a disease-causing microorganism dependent on sialic acid metabolism, wherein the microorganism comprises a pathogen capable of producing neuraminidase as a virulence factor, the method comprising administering to an animal in need thereof a therapeutically effective amount of a composition comprising one or more compounds, wherein one of the compounds comprises neuraminidase inhibitors.

The neuraminidase dependent infection can be selected from the group consisting of bacterial, fungal, yeast and protozoa.

The neuraminidase inhibitor is selected from the group consisting of zamanivir (Relenza), oseltamivir (Tamiflu), rimantadine, rimantadine hydrochloride, amantadine, ribavirin, and leaves and stem bark from Tamarindus indicus (T. indicus) and Combreton fragrans (C. fragrans), and the like, and any structural homologue of sialic acid, and any drug that are synthetic sialic acid analogs that can inhibit action of viral, bacterial, fungal, protozoan and eukaryotic neuraminidases.

Preferably, the neuraminidase inhibitor is any structural homologue of sialic acid.

Preferably, the neuraminidase inhibitor is oseltamivir.

### DESCRIPTION

According to the present invention, there is provided novel uses of selective neuraminidase inhibitors effective in shortening or stopping the pathophysiology of diseases involving one or more pathogens that require neuraminidase as a virulence factor.

### Neuraminidase

Neuraminidases, (also known as sialidases) are known to those skilled in the art as enzymes that have been identified in many viruses, bacteria, fungi, including yeast, and eukaryotes that cleave sialic acid moieties and can be involved in many functions in vivo. It has been shown that neuraminidases can play a significant role in the pathogenesis of infectious diseases, whose etiologic agents produce neuraminidase to cleave sialic acids in infected tissues to facilitate their ability to invade a host. It has been shown that there is a positive correlation between the level of production of sialidases and the virulence of various bacterial, fungal, including yeast, and protozoan strains. This virulence is further enhanced by different bacteria being able to produce more than one sialidase. Thus, many disease causing microorganisms possess a neuraminidase.

One example of a neuraminidase inhibitor that has been approved for the treatment of human influenza, is oseltamivir (TAMIFLU®, F. Hoffman-La Roche, Switzerland) and zanamivir (RELENZA®, Glaxo Wellcome, Inc., hereinafter referred to an RELENZA). Oseltamivir is a synthetic sialic acid analog that has been modified at the C4 position. Synthetic sialic acid analogs, such as oseltamivir have been demonstrated to inhibit the action of neuraminidases. Since their introduction in 1999, zanamivir and oseltmivir have been used successfully to treat human influenza A and B viral infections. In humans, neither zanamivir nor oseltamivir has been demonstrated to be effective in preventing serious influenza-related complications, such as bacterial or viral pneumonia or exacerbation of chronic diseases. Development of viral resistance to zanamivir and oseltamivir during treatment has been identified but does not appear to be frequent.

In some pathogens, including many enteric bacteria, neuraminidases typically are recognized as virulence factors. Neuraminidases cleave terminal sialic acid residues from cell surface molecules such as glycoproteins and glycolipids. As a result of this cleavage, internal sugar residues can be exposed that are normally protected and not available to pathogens. Neuraminidase activity can be particularly important for bacterial adhesion to mucosal surfaces. Mucous typically is highly sialylated and can be a major component of innate mucosal immunity. In mucosal diseases, commensal bacteria are separated from epithelial cells by a mucous barrier. Pathogenic bacteria have been shown to produce sialidases which can decrease the viscosity of the mucous and thus enable the bacteria to colonize on the epithelial cell membrane. Once in contact with the epithelial cell, a pathogen can become attached. With bacterial colonization and proliferation, there can be detachment and depletion of immunoglobin IgA. Bacterial endotoxins and exotoxins can be released resulting in local and distant tissue damage. Bacterial neuraminidases (sialidases) can cause the dissolution of the neuraminic acid located within the intercellular cement of the epithelial cells, allowing bacteria, their endotoxins, exotoxins and any environmental free sialic acid to enter the submucosa.

According to the present invention, it is known to those skilled in the art, the mechanism of hydrolyses of sialic acid compound during neuraminidase inhibition and pathogens that use sialic acid, is fully described and incorporated herein by reference in its entirety (Vrim et al., Microbiology and Molecular Biology Reviews, March 2004, p. 132-153).

A definitive role of neuraminidase activity in, for example, canine parvoviral infections, has not been established and it is thought that canine parvovirus does not have a neuraminidase in its genome. However, in one preferred embodiment of the present invention, it has been found that it is not essential for canine parvovirus to contain or utilize neuraminidases in order for them to enhance pathogenicity. Neuraminidases have been known to demonstrate enhanced pathogenicity in a synergistic fashion in several viral and bacterial superinfections involving mucosal surfaces. In some cases, for example, pneumococcal pneumonia secondary to influenza, viral neuraminidase activity enhanced the adhesion of the bacteria to the mucosal surface that resulted in increased bacterial invasion into tissues and resistant bacterial superinfection. Neuraminidases of bacterial origin alone are known as vitally important virulence factors.

According to the present invention, as used herein, the Theory of Biological Intervention states that one can treat infectious diseases by suppressing or inhibiting one of more of the pathogen's virulence factors, wherein the virulence factor can be neuraminidase.

In a preferred embodiment of the present invention, a neuraminidase inhibitor is given and results in remission of clinical signs, the pathogen(s) can be identified as being neuraminidase producers or dependent. This is because of the singular specificity of a neuraminidase inhibitor stopping the removal of sialic acids from the host cell's glycoprotein, glycolipids and polysaccharides.

The enzyme neuraminidase (sialidase) is a virulence factor produced and secreted by the infectious organism (regardless of genus or species) to remove neuramic (sialic acid) molecules from the host's (regardless of genus or species) cells and tissues.

According to the present invention, regardless of the genus or species of the organism and host, there is only one enzymatic reaction associated with the removal of sialic acid from the host's cells and tissues.

Preferably, neuraminidase inhibitors are homologues of each other regardless of their source. Every neuraminidase inhibitor is also a homolog of neuramic (sialic) acid and inactivates the pathogen's neuraminidase by combining with its terminal end designed to attach and remove the host's neuramic (sialic) acid. The inactivated neuraminidase is prevented from removal of neuramic (sialic)acid from the host, and thus prevents or limits the degree of infection.

Sialic acid is known to those skilled in the art as a 9—carbon compound.

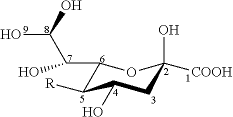

In a preferred embodiment of the present invention, neuraminidase (sialidase) is the enzyme produced by neuraminidase producing organisms to remove neuraminic (sialic) acid molecules from the host's cells and tissues. The site of neuraminidase attachment is structurally homologous to that of the targeted neuraminic (sialic) acid attached to the host's cells or tissues.

Preferably regardless of the genus and species of the host or pathogen, the enzymatic reaction where the pathogen's neuraminidase attaches and removes the host's sialic acid molecule is a singular identical reaction.

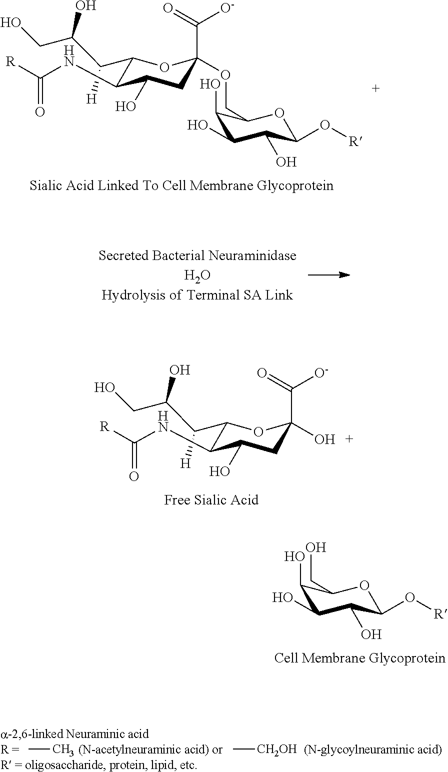

More preferably, Neuraminidase inhibitors combine with the active site of the pathogen's neuraminidase enzyme and in doing so, prevent the secreted neuraminidase from attaching to and removing the sialic acid molecules from the terminal ends of the host's glycoproteins, glycolipids and polysaccharides.

More preferably, regardless of the original source for a neuraminidase inhibitor, every neuraminidase inhibitor is a homolog to any other neuraminidase inhibitors and also to sialic acid. This homologous feature of neuraminidase inhibitors explains how to inhibit neuraminidases from different bacteria (Clostridium and Enterobacteria) by using neuraminidase inhibitors from 3 different plants (Illicium anisatum, Tamarindus indicus and Combretum fragrans).

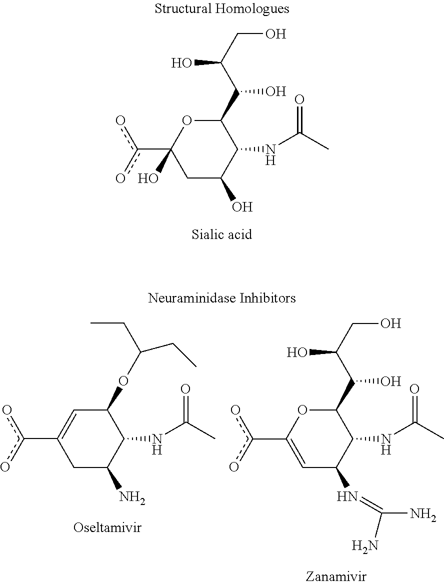

Preferably, the chemical and medical complexities associated with the current approach to infectious diseases are reduced to the inhibition of one simple enzymatic reaction by any one of a group of homologs of sialic acid called neuraminidase inhibitors.

The present invention provides the use of neuraminidase inhibitor to treat diseases involving neuraminidase dependent bacteria. Evidence to support this theory includes the following. It is known that the Fulani Pastoralists of rural Nigeria prevented blackleg infections in their cattle by feeding them the stem bark from two plants (Tamarindus indicus and Combretum fragrans). These plants contained neuraminidase inhibitors in their stem bark. Blackleg is a lethal disease in cows caused by a neuraminidase dependent bacteria Clostridium chauvoei. In one preferred embodiment of the present invention, it has been demonstrated that bacteria must be present in the disease causing microorganism, for example, parvovirus infection, to result in significant pathology. Typically, germ free animals do not demonstrate any of the clinical disease that is seen in normal animals when they are challenged with virulent parvovirus strains. The pathology is thought to be attributed to septicemia and endotoxemia and is believed to originate from enteric bacteria. Several enteric bacterial species are known to have neuraminidase activity including Escherichia coli, Campylobacterium, Salmonella, Shigella, Staphylococcus and Clostridium. From the list, at least two of these species, E. coli and Clostridium, have been associated with morbidity and mortality in dogs with parvovirus.

In addition, germ-free kittens and germ-free puppies when exposed to pathogenic strains of feline and canine parvovirus, did not develop any clinical signs. It is known to those skilled in the art that the commensal microflora of puppies contains neuraminidase dependent bacteria (Strep., E. coli, Staph., Clostridium, peptostreptococci, lactobacilli). According to the present invention, it has been shown that E. coli and Clostridium have been associated with morbidity and mortality in dogs with parvovirus. In addition, neuraminidases have been demonstrated to enhance pathogenicity in a synergistic fashion in some viral and bacterial superinfections involving mucosal surfaces. Still further, the role of sialic acid metabolism in commensal and pathogenic strains of neuraminidase dependent bacteria provides support for the methods used in accordance with the present invention. Further evidence supporting the role of neuraminidases in infectious diseases includes knowing that the histopathological lesions associated with canine parvoviral enteritis were typical of those created by bacterial septicemia and endotoxemia.

In addition, most if not all of these commensal bacteria produce neuraminidase in order to provide sialic acid to use in their metabolic pathways. When canine parvovirus exits an infected gastrointestinal(GI) epithelial cell, sialic acid is released into the GI tract. The commensal bacteria begins to colonize and proliferate and produce their own neuraminidase. This excess neuraminidase can provide additional sialic acid and can also dissolve the neuraminic acid in intercellular cement providing a portal to submucosal tissue. In addition, neuraminidase can also displace epithelial cells' IgA.

Interleukin-8 is known as a cytokine produced by many cell types including endothelial cells, fibroblast, respiratory epithelial cells, macrophages and PMNs. With the release of IL-8, the PMNs can mobilize intracellular sialidases that move to their cell membrane and causes the release of sialic acid from the membrane surface. The removal of sialic acid residues from the PMN's cell membrane allows them to attach to the endotheial cell wall and move by diapedesis towards the tissues containing high levels of IL-8.

High levels of neuraminidase can also stimulate dendritic cells to interact with macraphages. Both CD4 and CD8 lymphocytes can also be stimulated to produce Th1 and Th2 cytokines.

Thus, in a preferred embodiment of the present invention, canine and feline parvoviral enteritis is shown to be a superinfection (requiring a virus+neuraminidase dependent bacteria living on a mucous substrate). The pathology seen at necropsy is solely due to endo and exotoxins produced by the commensal bacteria turned pathogenic. In a preferred embodiment of the present invention, parvoenteritis is not known as a viral disease, but that the pathobiology is due to excess neuraminidase. Thus, when a neuraminidase inhibitor like TAMIFLU is administered early in the course of the disease or as a prophylactic, one can prevent the production of neuraminidase (sialidase) and one can prevent the commensal bacteria from becoming pathogenic.

As used herein, “neuraminidase dependent bacteria” includes “neuraminidase producing bacteria.”

In still yet another preferred embodiment of the present invention, the neuraminidase inhibitors can be used to target neuraminidase dependent bacterial infections, superinfections, and coinfections and not dependent on viral neuraminidase.

In still yet another preferred embodiment of the present invention, the neuraminidase inhibitors can be used to target neuraminidase dependent fungal, yeast and protozoan infections.

In one preferred embodiment of the present infection, “superinfection”, as used herein, means that an infection requires both virus and bacteria combined together to produce pathology more severe than either can alone.

“Coinfection”, as used herein, means two or more different bacterial strains together to produce pathology of a disease more severe than either can alone.

As used herein, the term “pathogen” refers to a microbe producing one or more virulence factors of which neuraminidase is one of. According to the present invention, the difference between pathogen and commensal bacteria is that commensal bacteria are not producing neuraminidase as virulence factors.

By the term “animal”, as used herein, can be any animal species, warm blooded or cold blooded, including a human being, who is infected with, or is likely to be infected with, microorganism producing disease, which are believed to be pathogenic. Animal includes but is not limited to human beings, canine, feline, bovine, equine, avian, porcine and any other species known to those skilled in the art, for example, sheep goats and rabbits.

The inhibitors of interest in this invention are neuraminidase dependent bacteria inhibitors. Of particular interest are those which are specific for the neuraminidase enzyme. Since many commensal and pathogenic bacteria also used environmental (hosts) sialic acids as sources of carbon, nitrogen, energy and amino sugars for cell wall synthesis, microbial sialic acid metabolism has been established as a virulence determinant in a range of infectious diseases. Both commensal and pathogen bacteria have been known to modify their cell membranes with sialic acids in order to masquerade as “self” to avoid, obvert or inhibit host's innate immunity. Dehydration at the sialic acid reducing ends, leading to formation of a planar structure known as N-acetyl-2,3-didehydro-2-deoxyneuraminic acid (diddeoxyNeu5Ac [Neu5Ac2en]. The flattened Neu5Ac2en ring mimics the transition state during hydrolysis of sialoglycoconjugates (Sia-O-acceptors) by glycosylhydrolases designated sialidases (synonymous with neuraminidase). Neu5Ac2en is typically known as a sialidase or neuraminidase inhibitor. In particular, a preferred group of inhibitors are those neuraminidase inhibitors which are similar in structure to Neu5Ac2en. For example, Neu5Ac2en has been known to those skilled in the art, to serve as the lead compound for synthesis of one of the most well known sialidase inhibitor, zanamivir (RELENZA). Most preferably, the neuraminidase inhibitors according to the present invention are those compounds that hydrolyze sialic acid.

In yet a more preferred embodiment of the present invention, the neuraminidase inhibitor is any compound that is a structural homologue of sialic acid.

“Structural homologue” as used herein, is defined as any compound that is corresponding in structure and origin but not necessary in function of sialic acid. As used herein, neuraminidase inhibitors are structural homologues to each other and of sialic acid.

“Neuraminidase dependent infections” as used herein, are based on the metabolism of sialic acid, which is dependent on one enzymatic reaction, and can be controlled using a neuraminidase inhibitor from any source, since neuraminidase inhibitors are homologues of each other and sialic acid. The enzymatic reaction in which neuraminidase initiates and controls the rate of a chemical reaction that involves the cleavage of glucosidic linkage between a sialic acid residue and a hexose or hexoamine residue at the non-reducing terminal of oligosaccharides in glycoproteins, glycoplipids and proteoglycans, and is a single specific enzymatic reaction that is not influenced by the animal species or sex. A “neuraminidase inhibitor”, as used herein, is any compound that is a homologue of sialic acid, and can inhibit this specific enzymatic reaction.

Specifically, with respect to “Theory of Biological Intervention” where one can treat infectious diseases by suppressing or inhibiting one ore more of the pathogen's virulence factors, it is known to those skilled in the art that protozoan (Tritrichomonas spp) and yeast (Candida spp), are neuraminidase producers and produce neuraminidase as a virulence factor.

In yet another preferred embodiment, the present invention also provides the use of neuraminidase inhibitor to treat, inhibit and prevent diseases involving neuraminidase as a virulence factor including but not limited to fungi, including yeast, protozoan and bacterial infections in which the pathogen is neuraminidase producer. Evidence to support this theory includes the following. Several fungal and protozoan species are known to have neuraminidase activity including Candida fumata for yeast; and Tritrichomonas foetus for protozoan.

Candida infections typically are reported in areas that contain a layer of mucin between the epithelial cells of the organ and lumen of the organ system. Historically, they can be very difficult to treat requiring months of therapy using different antifungal drugs.

Antifungal drugs are known to those skilled in the art to be toxic to the host's liver and kidney, and so any therapy that shortens the treatment period is significant.

The introduction of oseltamivir phosphate to suppress the pathogen's (Candida famata) production of neuraminidase greatly shortened the clinical disease from weeks to 72 hours. This case is an example of using the Theory of Biological Intervention in a clinical yeast/fungal infection. The Theory of Biological Intervention states that one can treat most infections by suppressing one of more of the pathogen's virulence factors. In this case, neuraminidase is a virulence factor of Candida famata (C. famata).

It is known to those skilled in the art that Candida is a genus of yeast. The use of the term Candida often refers to a complex with broad spectrum of symptoms, the majority which center around, for example, gastrointestinal distress, rashes, and sore gums. Other Candida species include but are not limited to C. famata; Candida albicans (C. albicans, also known to those skilled in the art as thrush); C. glabrata and C. rugosa.

Preferably, since bacteria, protozoan and fungi (yeast) produce neuraminidase as virulence factors, according to the “Theory of Biological Intervention”, protozoan, fungi, yeast, bacterial and viral infections can be treated, inhibited and prevented using a therapeutically effective amount of a composition comprising one or more compounds. Preferably, one of the compounds comprises neuraminidase inhibitors.

With respect to Candida spp, neuraminidase can degrade protective mucin layer in a gastro intestinal (GI) tract and allows Candida spp to attach to the tissues. Neuraminidase inhibitors of the present invention, can block the production of neuraminidase by Candida spp and suppress the Candida infection. Moreover, neuraminidase inhibitors (not limited to oseltamivir phosphate, for example) can be used in combination with an antifungal drug (such as Itraconazole, for example). Typical antifungal treatments typically can take months of various antifungal drugs, each with potential harm to the animal's liver and kidneys.

According to an embodiment of the invention, protozoan infections (such as Tritrichomonas spp) can be treated with neuraminidase inhibitors. Tritrichomonas spp preferably can include Tritrichomonas foetus (T. foetus). T. foetus typically is known to those skilled in the art as an obligate parasite of the bovine urogenital tract producing infection associated with inflammatory changes, abortion and infertility. Typically, although the two tritrichomonas have different habitats, both protozoans are known to use lectins with sialic acid specificity for adhesion to mucosal surfaces. Typically, the drug, Ronidazole is effective to treat T. foetus in cats. However, Ronidazole, a nitroimidazole, has many side effects and is known to be carcinogenic. Cats that have been given Ronidazole have been known to develop ataxia, nystagmus and behaviour changes. These signs of neurotoxicity have shown to be reversible when Ronidazole was discontinued.

One of the virulence factors produced by T. foetus is neuraminidase. Neuraminidase is an enzyme that when exposed to glycoproteins found in body tissues can release a nine carbon compound called neuramic or sialic acid. This compound typically is found in every living thing and therefore is known as a basic building block in nature. T. foetus secrets neuraminidase into the gastrointestinal (GI) lumen and it can dissolve the mucin layer between the GI contents and the GI epithelial cells. Neuraminidase also is known to denature IgA attached to the GI epithelial cell and also alters the GI epithelial cell membrane to allow T. foetus to colonize and reproduce by using the freed neuramic acid molecules.

Oseltamivir phosphate is a neuraminidase inhibitor that has been shown to inhibit neuraminidases produced by GI organisms in parvoenteritis. Given the choice of using ronidazole or bloody diarrhea for up to two years, it was decided to use oseltamivir phosphate to see what suppressing T. foetus' ability to secrete neuraminidase would do to the clinical course of this infection.

In a preferred embodiment the use of a neuraminidase inhibitor has been used to inhibit, treat and prevent neuraminidase dependent bacterial infections that are not viral generated dependent infections, from a disease causing microorganism dependent on sialic acid metabolism, comprising administering to an animal in need thereof a therapeutically effective amount of composition comprising one or more compounds, wherein one of the compounds comprises a neuraminidase inhibitor, wherein the neuraminidase inhibitor is any compound that is a structural homologue of sialic acid.

More preferably, structural homologue can defined as any compound that is corresponding in structure and origin but not necessary in function of sialic acid.

Also included are resistant bacteria where the pathogen can be a gram (+) rod bacteria and where potential drugs alone were ineffective (for example, the bacteria were resistant). Also included in an embodiment of the present invention are bacterial infections in the frontal sinus regions which can be difficult to treat.

In a preferred embodiment, the present invention provides the use of neuraminidase inhibitor to treat a neuraminidase dependent bacterial infection, selected from the group consisting of Enterococcus faecalis, (E. faecalis, which affects the sinuses), E. coli in the kidney Clostridium perfringens (Necrotic enteritis) and antibiotic resistant bacteria.

Preferably, the animal can include cold blooded animals and warm blooded animals. Animal includes but is not limited to human beings, canine, feline, bovine, equine, avian, porcine and any other species known to those skilled in the art, for example, sheep goats and rabbits.

In a preferred embodiment of the present invention, the use of a neuraminidase inhibitor has been used to treat necrotic enteritis (NE) in poultry. More preferably, the neuraminidase inhibitor comprises Tamerindus Indicus and Combretum fragrans.

In yet another preferred embodiment of the present invention, the use of a neuraminidase inhibitor has been used to treat feline Enterobacteria faecalis sinusitis. More preferably, the neuraminidase inhibitor is oseltamivir (TAMIFLU®).

According to a preferred embodiment of the present invention, Theory of Biological Intervention states that one can treat many infections by suppressing one or more of the pathogen's virulence factors. By suppressing one or more of the pathogen's virulence factors does not kill the organism, but does prevent the invasion of the host's body tissues while also denying or reducing the supply of carbon compounds normally used by the organism for energy and building blocks to reproduce and colonize on the host's tissues.

### Treatment

According to one embodiment of the present invention, an effective amount of compound, preferably a neuraminidase inhibitor can be administered to an animal. Typically, when a parvovirus infected animal presents symptoms such as vomiting/nausea and pain, traditional treatment involves administering fluids and cortisone for shock, antibiotics therapy and medicine for pain. In addition, anti-emetics can be administered to help alleviate nausea and vomiting.

The neuraminidase inhibitor can be administered in several ways: i) at the start of or during the course of the neuraminidase dependent bacterial infection, or some part thereof; or ii) at the start of or during the course of a superinfection infection or some part thereof; or iii) at the start of or during the course of a coinfection or some part thereof. In addition, the inhibitor can be administered prior to the onset of a neuraminidase dependent bacterial infection, superinfection or coinfection, and preferably continued for some period during the course of the bacterial infection, superinfection or coinfection. In a most preferred embodiment of the present invention, the neuraminidase inhibitor can be administered during the entire, or part of the length of a bacterial infection, a superinfection or a co-infection.

The neuraminidase inhibitor can also be administered in several ways: i) at the start of or during the course of the neuraminidase dependent bacterial, fungal, yeast and protozoan infection, or some part thereof; or ii) at the start of or during the course of a superinfection infection or some part thereof; or iii) at the start of or during the course of a coinfection or some part thereof. In addition, the inhibitor can be administered prior to the onset of a neuraminidase dependent bacterial, fungal, yeast and protozoan infection, superinfection or coinfection, and preferably continued for some period during the course of the bacterial, fungal, yeast and protozoan infection, superinfection or coinfection. In a most preferred embodiment of the present invention, the neuraminidase inhibitor can be administered during the entire, or part of the length of a bacterial, fungal, yeast and protozoan infection, a superinfection or a co-infection.

Most preferably, the neuraminidase inhibitor is administered within 48 hours of onset of first clinical signs.

By the term “an effective amount” is meant an amount of the compound in question which will in a majority of animals have either the effect that the disease caused by the pathogen is cured or, if the substance has been given prophylactically, the effect that the disease is prevented from manifesting itself. The term “an effective amount” also implies that the substance is given in an amount which only causes mild or no adverse effects in the animal to whom it has been administered, or that the adverse effects may be tolerated from a medical and pharmaceutical point of view in the light of the severity of the disease for which the substance has been given.

For the purposes of this invention, it is preferred to administer an effective amount of the neuraminidase inhibitor in an amount from about 0.6 mg/lb to 12 mg/lb, more preferably 0.3 mg/lb to 10 mg/lb, and most preferably 1 mg/lb of the active ingredient. Too high a dose of neuraminidase inhibitor can be toxic. Too low of a dose may not be effective enough to treat or prevent the neuraminidase dependent disease.

The neuraminidase inhibitor can be administered by any route. The route of administration of the substance could be any conventional route of administration, i.e. oral, intravenous, intramuscular, intradermal, subcutaneous etc. A preferred formulation will be the oral route; oral immediate release tablet or an oral controlled release tablet. For treatment of a disease caused by a microorganism, the neuraminidase inhibitor can be administered up to 6 times per day, though twice or once a day dosing regime is preferred. More preferably, 10 doses over a period of 5 days. Most preferably, 6 doses over a period of 3 days or until the animal's health improves.

In yet another preferred embodiment of the present invention, for prevention of a disease caused by a microorganism, the neuraminidase inhibitor can be administered once a day for 5 days. Typically, with animals infected with parvovirus, administering the neuraminidase inhibitor with the first dose will stop the vomiting. After the 2^{nd} dose, the diarrhea will cease. By the 6^{th} dose, most clinical signs of the infection will have ceased.

In one preferred embodiment, a composition can be administered to an animal, the composition comprising a compound. The compound preferably is a selective neuraminidase inhibitor. The neuraminidase inhibitor can be used alone or in combination with any other neuraminidase inhibitor. More preferably, the compound is a neuraminidase inhibitor which is selective towards neuraminidase dependent bacteria. Preferably, the neuraminidase inhibitor can be selected from the group consisting of zanamivir (RELENZA®, Glaxo Wellcome, Inc.), oseltamivir (TAMIFLU®, F. Hoffmann La Roche, Switzerland), rimantadine, rimantadine hydrochloride, amantadine, ribavirin, and leaves and stem bark from Tamarindus indicus (T. indicus) and Combreton fragrans (C. fragrans), and the like and any drug that are synthetic sialic acid analogs that can inhibit action of viral, bacterial, fungal, yeast, protozoan and eukaryotic neuraminidases. Preferably, the neuraminidase inhibitor is any compound that is a structural homologue of sialic acid. More preferably, the structural homologue is any compound that is corresponding in structure and origin, but not necessary in function, of sialic acid. Most preferably, the compound is a neuraminidase inhibitor that is oseltamivir. Oseltamivir (TAMIFLU®) is available from Roche Pharma™ AG (Switzerland). Alternatively, oseltamivir can be prepared according to the methods described in U.S. Pat. No. 5,763,483 to Bischofberger et al and U.S. Pat. No. 5,866,601 to Lew et al., the disclosures of which are hereby incorporated by reference. Preferably, the neuraminidase inhibitor comprises Tamerindus indicus and Combretum fragrans.

While the administration of neuraminidase inhibitor as the sole compound of the composition is most preferred, one or more of these neuraminidase inhibitors can be combined with other compounds for treating bacterial infections, fungal infections, yeast infections, protozoan infections, superinfections and coinfections. For example, a neuraminidase inhibitor could be co-administered with a treatment during the course of the neuraminidase dependent infection. Examples of drugs that can also be used in combination with one or more other compounds without limitation, are anti-infective agents and/or other agents used to treat other acute or chronic ailments which include, antimicrobial compounds (such as antibiotics), antifungal compounds, antiviral compounds, anticancer compounds, vitamins, trace metal supplements, or ionic buffers designed to maintain or correct proper ionic balance in blood or other tissues, such drugs are alpha and beta interferon, Inosine pranobex, moroxydine hydrochloride and the like. If antibiotics are used, preferably, the antibiotic is selected from the group consisting of penicillins, benzylpenicillin, amoxycillin, ampicillin, cephalosporins, erythromycin and co-trimoxazole.

Typically, Itraconazole can be used as an antifungal.

Appropriate dose ratio between a compound of the present invention and a second therapeutic compound for co-administration to an animal will be readily appreciated by those skilled in the art. Clearly, the combination therapies described herein are merely exemplary and are not meant to limit possibilities for other combination treatments or co-administration regimens.

### EXAMPLES

The following examples show the importance of neuraminidase dependent bacteria in mucosal infections in several animal species.

TABLE 1
Neuraminidase Dependent Bacteria and Veterinary Diseases

| Bacteria spp: | Dog | Cat | Cow | Pig | Horse | Avian | Other |
|---|---|---|---|---|---|---|---|
| Actinobacillus | + | + | + | + | + | | |
| Actinomyces | + | + | + | + | + | + | + |
| Aeromonas | + | | + | + | | + | + |
| Bacteroides | + | + | + | + | + | | + |
| Bordetella | + | + | | + | + | + | + |
| Brucella | + | + | + | + | + | | + |
| Campylobacter | + | + | + | + | + | + | + |
| Clostridium | + | + | + | + | + | + | + |
| Corynebacterium | + | + | + | + | + | + | + |
| Enterobacter | + | + | + | + | + | | |
| E. coli | + | + | + | + | + | + | + |
| Erysipelothrix | + | + | + | + | + | + | + |
| Fusobacterium | + | + | + | + | + | + | |
| Klebsiella | + | + | + | + | + | | |
| Pasturella | + | + | + | + | + | + | + |
| Mannheimia | | | + | | | | + |
| Peptostreptococcus | + | + | + | + | + | + | |
| Proteus | + | + | + | + | + | + | + |
| Pseudomonas | + | + | + | + | + | + | + |
| Rhodococcus | | + | | | + | | |
| Salmonella | + | + | + | + | + | + | + |
| Serratia | + | + | + | + | + | + | + |
| Shigella | + | + | + | + | + | | + |
| Staphlococcus | + | + | + | + | + | + | + |
| Streptococcus | + | + | + | + | + | + | + |
| Vibrio | | | | | | + | + |
| Haemophilus | + | + | + | + | + | + | + |
| Arcanobacterium | + | + | + | + | | | + |

Neuraminidase dependent bacteria are those known to use sialiac acid (neuraminic acid) either as a source for carbon, nitrogen, energy and amino acids for cell wall synthesis. This microbial sialic acid metabolism is known to be a virulence factor in a number of infectious diseases. Tables (9-14) representing specific diseases in the various species are included.

TABLE 2
Superinfections in Veterinary Medicine

| Species: | Disease | Virus | Bacteria | Other |
|---|---|---|---|---|
| Canine | Parvoviral Enteritis | Canine Parvovirus | Clostridium | Salmonella |
| | | CPV-2b > CPV-2a | E. coli | Peptostreptococcus |
| | | | Streptococcus | |
| | | | Staphylococcus | |
| | Tracheobronchitis | Canine Adenovirus-1 | Bordetella bronchiseptica | Streptococcus |
| | (Kennel Cough) | Canine Adenovirus-2 | | Pasturella |
| | | Canine Parainfluenza | | Pseudomonas |
| | | | | Klebsiella |
| | | | | E. coli |
| Feline | Parvoviral Enteritis | Feline Parvovirus | Clostridium | |
| | (Panleukopenia) | | E. coli | |
| | | | Streptococcus | |
| | | | Staphylococcus | |
| | | | Peptostreptococcus | |
| URI | Feline Rhinotracheitis | Feline Herpesvirus-1 | Bordetella bronchiseptica | Streptococcus |
| Complex | | | | Pasturella |
| | | | | Pseudomonas |
| | Feline Calicivirus | Feline Calicivirus | Bordetella bronchiseptica | Klebsiella |
| | | | | E. coli |
| | | | | Chlamydia |
| Bovine | Enzootic Pneumonia | Parainfluenza-3 (Pi-3) | Pasturella multocida | Mycoplasma dispar |
| | | Bovine Respiratory | Arcanobacterium | Mycoplasma bovis |
| | | Syncytial Virus (BRSV) | pyognes | Ureaplasma |
| | | Bovine Herpes -1 | Haemophilus somnus | Chlamydia |
| | | Reoviruses | E. coli | |
| | | Rhinoviruses | | |
| | Shipping Fever or | Pi-3 | Mannheimia haemolytica | Pasteurella multocida |
| | Pneumonic pasteurellosis | BRSV | | |
| | | BHV-1 | | |
| | Infectious Bovine | Bovine Herpes-1 | Mannheimia haemolytica | Pasteurella multocida |
| | Rhinotracheitis (IBR) | | | |
| | Bovine Viral Diarrhea | BVD-1 | Clostridium | |
| | | BVD-2 | E. coli | |
| | | | Streptococcus | |
| | | | Staphylococcus | |
| | | | Peptostreptococcus | |
| Porcine | Swine Influenza | Swine Influenza-A | Pasturella multocida | Arcanobacterium |
| | | | | pyogenes |
| | | | | Haemophilus |
| | Inclusion Body Rhinitis | Porcine Cytomegalovirus | Bordetella bronchiseptica | |
| | (Atrophic Rhinitis) | (PCMV) | Pasturella multocida | |
| | Porcine Reproductive | PRRSV | Streptococcus suis | |
| | and Respiratory Syndrome | | Haemophilus parasuis | |
| | (PRRS) | | Arcanobacterium suis | |
| | | | E. coli | |
| | Transmissible | TGEV | E. coli | Streptococcus |
| | Gastroenteritis (TGE) | | Clostridium | Staphlococcus |
| Equine | Equine Influenza | EIV-1 | Streptococcus | |
| | | EIV-2 | zooepidermicis | |
| | | | Staphlococcus aureus | |
| Avian | | | | |
| Chicken | Infectious Bronchitis | IBV | RespiratoryE. coli | |
| Turkey | Hemorrhagic Enteritis | Adenovirus | EnteropathicE. coli | |
| | | | EnteropathicE. coli | |
| | Poult Enteritis | Coronavirus | EnteropathicE. coli | |
| Ovine | Pneumonic Pasturellosis | ORSV | Mannheimia haemolytica | Pasturella multocida |
| | | Pi-3 | | |
| | | Adenovirus | | |
| | | Reovirus | | |

Table 2 represents a partial list of infectious diseases in veterinary medicine known to be superinfections. Superinfections are those diseases requiring at least 2 different infectious microbes, that together produce a disease that neither are capable of doing alone. In these cases, one or more virus are associated with one or more neuraminidase dependent bacteria.

Feline Parvovirus and Upper Respiratory Complex and canine Parvoviral Enteritis and Tracheobronchitis have proven to be responsive to neuraminidase inhibitors. There is no reason, the other superinfections will not respond in the same manner.

TABLE 3
Parvo Cases at Chihuahua Kennel

| Case | | | | | IV | Tamiflu | Days to |
|---|---|---|---|---|---|---|---|
| Number | Town State | Breed | Age | Parvo Test | Drugs | 1 mg/lb | Recover |
| 1 | DC County Loving | Chihuahua | 6 wks | (+) | Yes | None | Died |
| 2 | Kennel, Purdon, TX | Chihuahua | 6 wks | No | Yes | None | Died |
| 3 | | Chihuahua | 6 wks | No | Yes | None | Died |
| 4 | | Chihuahua | 6 wks | No | Yes | None | Died |
| 5 | | Chihuahua | 6 wks | No | Yes | None | Died |
| 6 | | Chihuahua | 6 wks | No | Yes | None | Died |
| 7 | | Chihuahua | 6 wks | No | Yes | None | Died |
| 8 | | Chihuahua | 6 wks | No | Yes | None | Died |
| 9 | | Chihuahua | 6 wks | No | Yes | None | Died |
| 10 | Changed Veterinarian | Chihuahua | 6 wks | (+) | None | AM/PM | 5 |
| 11 | | Chihuahua | 6 wks | No | None | AM/PM | 3 |
| 12 | | Chihuahua | 6 wks | No | None | AM/PM | 3 |
| 13 | | Chihuahua | 6 wks | No | None | AM/PM | 3 |
| 14 | | Chihuahua | 6 wks | No | None | AM/PM | 3 |
| 15 | | Chihuahua | 6 wks | No | None | AM/PM | 3 |
| 16 | | Chihuahua | 6 wks | No | None | AM/PM | 3 |
| 17 | | Chihuahua | 6 wks | No | None | AM/PM | 3 |
| 18 | | Chihuahua | 6 wks | No | None | AM/PM | 3 |
| 19 | | Chihuahua | 6 wks | No | None | AM/PM | 3 |
| 20 | | Chihuahua | 6 wks | No | None | AM/PM | 3 |
| 21 | Exposed—Preventive | Chihuahua | 6 wks | No | None | AM | Healthy |
| 22 | | Chihuahua | 6 wks | No | None | AM | Healthy |
| 23 | | Chihuahua | 6 wks | No | None | AM | Healthy |
| 24 | | Chihuahua | Adult | No | None | AM | Healthy |
| 25 | | Chihuahua | Adult | No | None | AM | Healthy |
| 26 | | Chihuahua | Adult | No | None | AM | Healthy |
| 27 | | Chihuahua | Adult | No | None | AM | Healthy |
| 28 | | Chihuahua | Adult | No | None | AM | Healthy |

Table 3 represents 28 Chihuahua dogs and puppies that experienced an outbreak of canine parvoviral enteritis within their kennel. The initial treatment lasted one week and was consistent with traditional therapy. (IV fluids, antibiotics and antiemetics). During the first week, 9 puppies died and a second veterinarian was consulted.

The second veterinarian removed all IV treatment and started oral TAMIFLU and AmoxiDrops on 11 puppies. This treatment was administered by the kennel staff with the veterinarian consulting by phone. All puppies survived with the new protocol.

The exposed dogs received 1 mg/lb of TAMIFLU once a day for 5 days. Although exposed, these dogs remained healthy.

TABLE 4
Canine Parvo Cases at Sandcastle Kennels

| Case | | | | | Parvo | IV | Tamiflu | Days to |
|---|---|---|---|---|---|---|---|---|
| Number | Town State | Breed | Age | | Test | Drugs | 1 mg/lb | Recover |
| 1 | Foyil Oklahoma | Cocker | 6 | wk | (+) | Yes | None | Died |
| 2 | | Cocker | 6 | wk | No | Yes | None | Died |
| 3 | | Cocker | 6 | wk | No | Yes | None | Died |
| 4 | | Cocker | 6 | wk | No | Yes | None | Died |
| 5 | | Cocker | 6 | wk | No | Yes | None | Died |
| 6 | | Cocker | 8 | wk | (+) | Yes | None | Died |
| 7 | | Cocker | 8 | wk | No | Yes | None | Died |
| 8 | | Cocker | 8 | wk | No | Yes | None | Died |
| 9 | | Cocker | 8 | wk | No | Yes | None | Died |
| 10 | Changed Veterinarian | Cocker | 6 | wk | (+) | None | AM/PM | 3 |
| 11 | | Cocker | 6 | wk | No | None | AM/PM | 5 |
| 12 | | Cocker | 8 | wk | (+) | None | AM/PM | 3 |
| 13 | | Cocker | 8 | wk | No | None | AM/PM | 5 |
| 14 | | Cocker | 8 | wk | No | Yes | AM/PM | 5 |
| 15 | | Cocker | 10 | wk | (+) | None | AM/PM | 3 |
| 16 | | Cocker | 11 | wk | No | None | AM/PM | 3 |
| 17 | | Cocker | 12 | wk | (+) | None | AM/PM | 4 |
| 18 | | Cocker | 12 | wk | No | None | AM/PM | 5 |
| 19 | | Cocker | 12 | wk | No | None | AM/PM | 3 |
| 20 | | Cocker | 12 | wk | (+) | Yes | AM/PM | 4 |
| 21 | | Cocker | 14 | wk | (+) | None | AM/PM | 5 |
| 22 | Exposed—Preventive | Cocker | 7 | month | No | None | AM | Healthy |
| 23 | | Cocker | 7 | month | No | None | AM | Healthy |
| 24 | | Cocker | 10 | month | No | None | AM | Healthy |
| 25 | | Cocker | 10 | month | No | None | AM | Healthy |

Table 4 represents of 25 cocker spaniel dogs and puppies that experienced an outbreak of canine parvoviral enteritis within their kennel. The initial treatment lasted one week and was consistent with traditional therapy consisting of IV fluids and antibiotics, antiemetics and steroids. During this period of time, 9 puppies died, and a second veterinarian was consulted.

The second veterinarian removed all IV treatment and oral TAMIFLU and sulfadimethoxine/ormetoprim (antibiotic) were the only drugs administered to 11 of the puppies. The 12th puppy was taken to the veterinarian's clinic and received IV therapy. Those puppies remaining at the kennel were treated by the kennel staff.

The exposed dogs received 1 mg/lb of TAMIFLU once a day for 5 days and did not develop canine parvoviral enteritis.

TABLE 5
Canine Parvoviral Enteritis Treated With Tamiflu

| Case | | | | | Parvo | IV | Tamiflu | Days to |
|---|---|---|---|---|---|---|---|---|
| Number | Town State | Breed | Age | | Test | Drugs | 1 mg/lb | Recover |
| 1-10 | Pinehurst, NC | Mix | 6-12 | wks | (+) | None | AM/PM | 3 to 5 |
| 11 | Griffin, GA | Mix | 11 | wks | (+) | None | AM/PM | 3 |
| 12 | | Mix | 14 | wks | (+) | Yes | AM/PM | 2 |
| 13 | | Mix | 14 | wks | (+) | Yes | AM/PM | 2 |
| 14 | Rockford, IL | GSH | 8 | wks | (+) | Yes | AM/PM | 3 |
| 15 | Clayton, NC | JRT | 7 | months | (+) | Yes | AM/PM | 5 |
| 16 | Carthage, NC | Mix | 19 | wks | (+) | None | AM/PM | 3 |
| 17 | | Mix | 20 | wks | (+) | None | AM/PM | 4 |
| 18 | Apple Valley, CA | Beagle | Pup | | (+) | Yes | AM/PM | 3 |
| 19 | Millington, TN | GSH | Pup | | (+) | Yes | AM/PM | 2 |
| 20 | Douglas, GA | Basset | 12 | wks | (+) | Yes | AM/PM | 2 |
| 21 | | It. Greyh. | 12 | wks | (+) | Yes | AM/PM | 3 |
| 22 | | Boxer | 12 | wks | (+) | Yes | AM/PM | 3 |
| 23 | Canton, OH | Rotti-x | 6 | months | (+) | Yes | AM/PM | 2 |
| 24 | Griffin, GA | Mix | Pup | | (+) | Yes | AM/PM | 3 |
| 25 | | Mix | Pup | | (+) | Yes | AM/PM | 3 |
| 26 | | Mix | Pup | | (+) | Yes | AM/PM | 2 |
| 27 | Salisbury, MD | Pit Bull-x | 6 | wks | (+) | None | AM/PM | 2 |
| 28 | Redford, MI | Pit Bull | 9 | months | Corona | Yes | AM/PM | 4 |
| 29 | Grand Rapids, MI | Mix | Pup | | (+) | Yes | AM/PM | 3 |
| 30 | | Mix | Pup | | (+) | Yes | AM/PM | 3 |
| 31 | | Mix | Pup | | (+) | Yes | AM/PM | 3 |
| 32 | Bend, OR | Bost. Terr. | 6 | months | (+) | Yes | AM/PM | 2 |
| 33 | Mishawaka, IN | Eng. Sett. | 14 | wks | None | Yes | AM/PM | 4 |
| 34 | Vancouver, WA | Rotti | 8 | wks | (+) | Yes | AM/PM | 5 |
| 35 | Atlanta, GA | Mix | 7 | wks | (+) | Yes | AM/PM | 3 |
| 36 | Jonesboro, AR | Min. Sch. | 6 | months | (+) | Yes | AM/PM | 2 |
| 37 | | Beagle-x | 5 | months | (+) | Yes | AM/PM | 4 |
| 38 | Columbia, MO | Mix | Pup | | (+) | Yes | AM/PM | 3 |
| 39 | Ocoee, FL | Shar Pei | 4 | months | (+) | None | AM/PM | 2 |
| 40 | Mishawaka, IN | Mix | 14 | wks | (weak) | Yes | AM/PM | 4 |
| 41 | | Mix | 4 | months | (+) | Yes | AM/PM | 3 |
| 42 | | Mix | 12 | wks | (+) | Yes | AM/PM | 3 |
| 43 | Atlanta, GA | Mix | 10 | wks | (+) | None | AM/PM | 3 |
| 44 | | Gold. Ret. | 8 | wks | (+) | Yes | AM/PM | 2 |
| 45 | Los Angeles, CA | St. Ber. mix | 10 | wks | (+) | None | AM/PM | 2 |
| 46-48 | Garden City, KS | Lab | 6 | months | (+) | None | AM/PM | 3 |
| | (exposed) | B. CollieX | 12 | weeks | (weak) | None | AM/PM | Normal |
| | | B. CollieX | 12 | weeks | (+) | None | AM/PM | 4 |

| | |
|---|---|
| Summary: | |
| States | 15 |
| DVMS | 20 |
| Puppies | 48 |

Table 5 represents 48 individual cases of Canine Parvoviral Enteritis treated with 1 mg/lb TAMIFLU AM/PM for 10 treatments. Cases posted VIN's Infectious Dz Board by 20 veterinarians practicing in 15 states.

TABLE 6
Feline Parvoenteritis Treated with Tamiflu

| Case | | | | | Parvo | IV | Tamiflu | Days to |
|---|---|---|---|---|---|---|---|---|
| Number | Town State | Breed | AgeSex | | Test | Drugs | 1 mg/lb | Recover |
| 1 | Smithfield, NC | Siamese | 5 | M/m | (+) | Yes | AM/PM | 2 |
| 2 | | Siamese | 5 | M/fem | (+) | Yes | AM/PM | 3 |
| 3 | Alberta, Canada | DSH | 14 | wk/m | no WBCs | Yes | AM/PM | 3 |
| 4 | (Exposed) | DSH | 20 | wk/fem | condomate | None | AM | Normal |
| 5 | Phoenix, AZ | DSH | 10 | wk/fem | (+) | SQ fluids | AM/PM | 4 |
| 6 | | DSH | 10 | wk/fem | (+) | SQ fluids | AM/PM | 4 |

Table 6 represents 5 cases of Feline Parvoviral Enteritis with TAMIFLU at 1 mg/lb AM/PM for 10 treatments. One kitten exposed, remained normal when given TAMIFLU at 1 mg/lb once a day for 5 days.

TABLE 7
Raccoon Parvoenteritis/Distemper Treated with Tamiflu

| Case | | | | | | Tamiflu | Days to |
|---|---|---|---|---|---|---|---|
| Number | Town State | Breed | Age | Parvo Test | IV Drugs | 1 mg/lb | Recover |
| 1 | Hudson, IL | Raccoon | Adult/Male | none | none | AM/PM | 3 |
| 2 | | Raccoon | Adult/Fem | none | none | AM/PM | 3 |
| 3 | Chiefland, FL | Raccoon | Adult | none | none | AM/PM | 3 |
| 4 | | Raccoon | Adult | none | none | AM/PM | 3 |
| 5 | | Raccoon | Adult | none | none | AM/PM | 3 |

Table 7 represents 5 raccoons treated with TAMIFLU at 1 mg/lb given every 12 hrs for 10 treatments. Treatment administered by civilian rehabbers at their homes. Granules mixed with pancake syrup.

Raccoons represent the 5th species (cow, dog, cat, mice) in which a neuraminidase inhibitor has been successful in treating or preventing a disease associated with neuraminidase dependent bacteria. Before using TAMIFLU, the hemorrhagic gastroenteritis (Parvo) in raccoon was 100% fatal. While the numbers are small they are significant as they prove the pathobiology seen in hemorrhagic gastroenteritis of raccoon is neuraminidase driven. Treatment was administered by untrained lay personnel at the rehab centers.

TABLE 8
Canine Kennel Cough Cases Treated With Tamiflu

| Case | | | | | Oral | Cough | Tamiflu | Days to |
|---|---|---|---|---|---|---|---|---|
| Number | Town State | Breed | Age | | Antibiotic | Suppressant | AM/PM | Recover |
| Holding Kennels for Pet Stores | | | | | | | 1 mg/lb | |
| 1-175 | Lynbrook, NY | Mixed | 8-12 | wks | Doxy | None | AM/PM | 3-5 days |
| 1-65 | New Hyde Park, NY | Mixed | 8-12 | wks | Doxy | None | AM/PM | 3-5 days |
| 1-60 | Lawrence, NY | Mixed | 8-12 | wks | Doxy | None | AM/PM | 3-5 days |
| Racing Greyhounds at Race Tracks | | | | | | | 1 mg/lb | |
| 1, 2, 3 | Miami, Florida | Greyhound | 1.5-4 | yr. | None | None | AM/PM | 3-5 days |
| 1-46 | Group A | Greyhound | 1.5-4 | yr. | Doxy | Dextromet | none | No Change |
| 1-46 | Group B | Greyhound | 1.5-4 | yr. | Cephalexin | Torbutrol | none | No Change |
| 1-47 | Group C | Greyhound | 1.5-4 | yr. | Clamamox | Hycodan | none | No Change |

***After 5 days, DVM stopped antibiotics and cough suppressants and started Tamiflu

| 1-46 | Group A | Greyhound | 1.5-4 | yr. | None | None | AM/PM | 3-5 days |
|---|---|---|---|---|---|---|---|---|
| 1-46 | Group B | Greyhound | 1.5-4 | yr. | None | None | AM/PM | 3-5 days |
| 1-47 | Group C | Greyhound | 1.5-4 | yr. | None | None | AM/PM | 3-5 days |
| 1-70 | Kan. City, Kansas | Greyhound | 1.5-4 | yr. | Doxy | None | AM/PM | 3-5 days |
| ***Track Veterinarian had to use 0.5 mg/lb due to cost | | | | | | | 0.5 mg/lb | |
| 1-72 | Mobile, Alabama | Greyhound | 11.5-4 | yr. | Pen—G | None | (+) | 5-10 days |

Infectious Canine Tracheobronchitis (ICT) or Kennel Cough is a highly infectious superinfection spread by aerosol droplets. The 3 holding kennels represent the first attempt at a herd health plan. The sick dogs were given TAMIFLU at 1 mg/lb AM/PM for 5 days. They recovered in 3-5 days. Those not showing clinical signs and any new puppy entering the kennel were given 1 mg/lb once a day for 5 days. This program reduced illness to below 5 percent, and cost of veterinary care by over 75%.

Kennel Cough outbreaks at Greyhound racing tracks result in the tracks being closed. In Miami, a total of 142 dogs became infected with ICT. They were separated into 4 groups: Group A, B and C received a different combination of antibiotic/cough suppressant. Three dogs were given TAMIFLU at 1 mg/lb AM/PM for 10 treatments. Groups A, B and C's clinical course was unchanged after 5 days of conventional therapy. Started TAMIFLU, and dogs recovered in 3-5 days.

The Miami experiment was the basis for treatment during a similar ICT outbreak at a Kansas City track.

Cost of TAMIFLU was a factor during an ITC outbreak in Mobil, Ala. They The DVM decided to give half the recommended dose (0.5 mg/lb). The results were better than conventional, but longer than when the recommended dose is used. This trial demonstrates that the response is dose related.

TABLE 9
Neuraminidase Dependent Bacteria and Canine Diseases

| Bacteria spp: | Respiratory | Urogenital | Gastrointestinal | Other |
|---|---|---|---|---|
| Actinomyces | Pyothorax | | | Deep Wounds |
| | Peritonitis | | | |
| Aeromonas | | | | Septicemia |
| Bacteroides | | | | Bone Infect. |
| Bordetella | Kennel Cough | | | |
| | Distemper | | | |
| | Upper Resp. Infect. | | | |
| Brucella | | Abortion | | |
| | | Infertility | | |
| Campylobacter | | | Gastroenteritis | |
| Clostridium | | | Gastroenteritis | Tetnus |
| | | | Parvoenteritis | Botulism |
| Corynebacterium | | | | |
| Enterobacter | | | | |
| E. coli | Upper Respiratory | Pyometra | Colibacillosis | |
| | Pneumonia | Mastitis | Parvoenteritis | |
| | | Renal Infections | | |
| | | Cystitis | | |
| Erysipelothrix | | | | Endocarditis |
| Fusobacterium | | | | |
| Klebsiella | Upper Respiratory | | | |
| | Pneumonia | | | |
| | | Cystitis | | |
| Pasturella | Upper Respiratory | | | |
| | Pneumonia | | | |
| Peptostreptococcus | | | | Abscesses |
| Proteus | | Upper and Lower | Gastroenteritis | Otitis |
| | | Urinary Tract | | |
| Pseudomonas | Upper Respiratory | Pyometra | | Otitis |
| | Pneumonia | Cystitis | | Dermatitis |
| Rhodococcus | | | | |
| Salmonella | | | Gastroenteritis | |
| Serratia | | | | |
| Shigella | | | Gastroenteritis | |
| Staphlococci | Upper Respiratory | Pyometra | | Otitis |
| | Pneumonia | Mastitis | | Dermatitis |
| | | Cystitis | | |
| Streptococci | Pneumonia | Pyometra | Parvoenteritis | Septicemia |
| | | Mastitis | | Puppy Strangles |
| | | Cystitis | | |
| Haemophilus | | | | |
| Arcanobacterium | | | | |

Table 9 is a partial listing of known neuraminidase dependent bacteria and the infectious diseases associated with them in the dog.

TABLE 10
Neuraminidase Dependent Bacteria and Feline Diseases

| Bacteria spp: | Respiratory | Urogenital | Gastrointestinal | Other |
|---|---|---|---|---|
| Actinomyces | Pyothorax | | | Abscess |
| | Peritonitis | | | |
| Aeromonas | | | | |
| Bacteroides | Emphyema | | | Abscess |
| Bordetella | Upper | | | |
| | Respiratory | | | |
| | Pneumonia | | | |
| Brucella | | | | |
| Campylobacter | | | Gastroenteritis | |
| Clostridium | | | Gastroenteritis | Tetnus |
| | | | Panleukopenia | |
| Corynebacterium | | | | |
| Enterobacter | | | | |
| E. coli | | Pyometra | Colibacillosis | |
| | | Mastitis | Panleukopenia | |
| | | Renal | | |
| | | Infections | | |
| | | Cystitis | | |
| Erysipelothrix | | | | |
| Fusobacterium | | | | |
| Klebsiella | Upper | | | |
| | Respiratory | | | |
| | Pneumonia | | | |
| | | Cystitis | | |
| Pasturella | Upper | | | |
| | Respiratory | | | |
| | Pneumonia | | | |
| Peptostrepto- | | | | |
| coccus | | | | |
| Proteus | | | | Otitis |
| Pseudomonas | Upper | Pyometra | | Otitis |
| | Respiratory | | | |
| | Pneumonia | Cystitis | | Abscess |
| Rhodococcus | Pyothorax | | | Abscess |
| Salmonella | | | Gastroenteritis | |
| Serratia | | | | |
| Shigella | | | Gastroenteritis | |
| Staphlococci | Upper | Pyometra | | Otitis |
| | Respiratory | | | |
| | Pneumonia | Mastitis | | Dermatitis |
| | | Cystitis | | |
| Streptococci | Pneumonia | Pyometra | Panleukopenia | Septicemia |
| | | Mastitis | | |
| | | Cystitis | | |
| Haemophilus | | | | |
| Arcanobacterium | | | | |

Table 10 is a partial listing of known neuraminidase dependent bacteria and the infectious diseases associated with them in the cat.

TABLE 11
Neuraminidase Dependent Bacteria and Bovine Diseases

| Bacteria spp: | Respiratory | Urogenital | Gastrointestinal | Other |
|---|---|---|---|---|
| Actinomyces | Pneumonia | Mastitis | | “Lumpy Jaw” |
| | | Endometritis | | Arthritis |
| | | Umbilical Infections | | Endocarditis |
| | | Seminal Vesiculitis | | Abscess |
| Aeromonas | | Mastitis | | |
| Bacteroides | | Mastitis | Gastroenteritis | Foot Rot |
| | | | | Osteomyelitis |
| Brucella | | Abortion | | |
| | | Orchitis | | |
| Campylobacter | | Epizootic Infertility | | |
| | | Embryonic Death | | |
| | | Abortion | | |
| Clostridium | | Gangrenous Mastitis | Enterotoxaemia | Tetanus |
| | | | | Botulism |
| | | | | Blackleg |
| | | | | Malignant Edema |
| | | | | Gas Gangrene |
| | | | | Bacillary Haemoglobinuria |
| Corynebacterium | | Pyelonephritis | | |
| | | Cystitis | | |
| | | Mastitis | | |
| Enterobacter | | Coliform Mastitis | | |
| E. coli | | Mastitis | “White Scours” | Septicemia |
| | | | Colibacillosis | Joint III |
| Fusobacterium | Calf Diphtheria | Mastitis | Liver Abscess in Feedlot | |
| | | Metritis | Hepatic Necrobacillosis | |
| Klebsiella | | Mastitis | | |
| Pasturella | “Shipping Fever ” | | | Hemorrhagic |
| | “Enzootic Pneumonia” | | | Septicemia |
| | | | | Fibrogranulomatous Disease |
| Peptostreptococcus | | Summer Mastitis | | |
| Proteus | | | Enteritis | |
| Pseudomonas | Focal Pneumonia | Mastitis | Enteritis | Dermatitis |
| | | Uterine Infections | | Abscess |
| | | | | Arthritis |
| Salmonella | | Abortion | Enteritis | Septicaemia |
| | | | | Meningitis |
| | | | | Joint III |
| | | | | Dry Gangrene |
| Serratia | | Mastitis | | in Calves |
| Staphlococci | | Mastitis | | |
| | | Udder impetigo | | |
| Streptococci | | Mastitis/Metritis | | |
| Haemophilus | Pneumonia | | | |
| Arcanobacterium | Pneumonia | Mastitis | Liver Abscess | Foot Rot |

Table 11 is a partial listing of known neuraminidase dependent bacteria and the infectious diseases associated with them in the cow.

TABLE 12
Neuraminidase Dependent Bacteria and Porcine Diseases

| Bacteria spp: | Respiratory | Urogenital | Gastrointestinal | Other |
|---|---|---|---|---|
| Actinomyces | Pneumonia | Pyogranulomatous | | Arthritis |
| | | Mastitis | | Lymphadenitis |
| | | Endometritis | | |
| | | Umbilical Infections | | |
| | | Seminal Vesiculitis | | |
| Aeromonas | | | Diarrhea | |
| Bacteroides | | | Diarrhea in Piglets | Abscess |
| Bordetella | Atrophic Rhinitis | | | |
| | Bronchopneumonia in | | | |
| | Young Piglets | | | |
| Brucella | | Abortion | | Arthritis |
| | | Orchitis | | Spondylitis |
| | | Infertility | | |
| Campylobacter | | Intestinal Adenomatosis | Diarrhea | |
| Clostridium | | | | Tetnus |
| | | | | Botulinum |
| | | | | Black Leg |
| | | | | Maligant Edema |
| | | | | Hemorrhagic Enterotoxemia |
| Corynebacterium | | Pyelonephritis | | |
| Enterobacter | | Mastitis-Metritis | | |
| | | Agalactia Complex(MMA) | | |
| E. coli | Associated with PRRSV | Mastitis | Neonatal Diarrhea | Piglet |
| | | Mastitis-Metritis | Colisepticemia | Meningitis |
| | | Agalactia Complex(MMA) | Weaning Enteritis | Sudden |
| | | | | Edema/death |
| Erysipelothrix | | Acute Abortion | | “Diamond Skin Disease” |
| | | | | Vegetative Endocarditis |
| | | | | Polyarthritis |
| Fusobacterium | “Bull-Nose” | | Necrotic Enteritis | Liver Abscess |
| Pasturella | Pneumonia Assoc. w/PRRSV | | | |
| | Atrophic Rhinitis | | | |
| Peptostreptococcus | | | | |
| Pseudomonas | Respiratory Infections | Abortion | Enteritis | Otitis |
| | | | | Arthritis |
| Rhodococcus | | | | Cervical Lymphadenitis |
| Salmonella | | | Hog Cholera | |
| | | | Chronic Enteritis | Septicemia |
| Serpulina | | | Swine Dysentery | |
| Staphlococci | | Mastitis | | Exudative Epidermitis or |
| | | Endometritis | | Greasy Pig Disease |
| | | Udder Impetigo | | |
| Streptococci | Rhinitis, Pneumonia | | | Lymphadenitis |
| | assoc. w/Porcine Reproductive | | | Arthritis |
| | and Respiratory Syndrome | | | |
| Haemophilus influenzae | Porcine Reproductive and | | | |
| | Respiratory Syndrome | | | |
| Arcanobacterium | Pneumonia | | | |

Table 12 is a partial listing of known neuraminidase dependent bacteria and the infectious diseases associated with them in the pig.

TABLE 13
Neuraminidase Dependent Bacteria and Equine Diseases

| Bacteria spp: | Respiratory | Urogenital | Gastrointestinal | Other |
|---|---|---|---|---|
| Actinomyces | | | | Poll Evil |
| | | | | Fistulous Withers |
| Aeromonas | | | | |
| Bacteroides | | | Diarrhea in Foals | Osteomylitis |
| | | | | Buccal Cavity Lesions |
| Bordetella | Respiratory Infections | | | |
| Brucella | | | | Poll Evil |
| | | | | Fistulous Withers |
| Campylobacter | | | | |
| Clostridium | | | Enteritis | Tetanus |
| | | | | Botulism |
| Corynebacterium | | | | Ulcerative Lymphangitis |
| Enterobacter | | Metritis | | |
| E. coli | | | Enteritis | |
| Erysipelothrix | | | | |
| Fusobacterium | | | | “Thrush” involving the frog |
| Klebsiella | Pneumonia in Foals | Metritis | | Abscess |
| | | Cervicitis | | |
| Pasturella | Respiratory Infections | | | |
| | Pneumonia | | | |
| Peptostreptococcus | | | | |
| Proteus | | Kidney infections | | |
| | | Cystitis | | |
| Pseudomonas | Lung Abscesses | Metritis | | Eye Infections |
| | Glanders | | | Lymphangitiswith ulcers |
| | | | | along lymphatics(Farcy) |
| Rhodococcus | Bronchopneumonia | | | |
| Salmonella | | Abortion | Sever Enteritis | Septicemia |
| Serratia | | | | |
| Shigella | | | | |
| Staphlococci | | Mastitis | | |
| | | Botryomycosis after | | |
| | | Castration | | |
| Streptococci | Pneumonia | Endometritis | | Foal Lymphangitis |
| | | Mastitis | | Abscess |
| | | Abortion | | Strangles |
| | | Navel Infections | | Purpura Hemorrhagica |
| | | Genital Infections | | |
| Haemophilus | | | | |
| Arcanobacterium | | | | |

Table 13 is a partial listing of known neuraminidase dependent bacteria and the infectious diseases associated with them in the horse.

TABLE 14
Neuraminidase Dependent Bacteria and Avian Diseases

| Bacteria spp: | Respiratory | Urogenital | Gastrointestinal | Other |
|---|---|---|---|---|
| Actinobacillus | | | | |
| Actinomyese | | | | |
| Aeromonas | | | | Septicemia |
| Bacteroides | | | | |
| Bordetella | Turkey Coryza | | | |
| | Rhinotracheitis | | | |
| | Sinusitis | | | |
| Campylobacter | | | Avian Vibrionic Hepatitis | |
| Clostridium | | | Necrotic Enteritis | Tetanus |
| | | | Ulcerative Enteritis | Botulism |
| | | | | Necrotic Dermatitis |
| | | | | “Struck” |
| Corynebacterium | | | | |
| Enterobacter | | | | |
| E. coli | Airsacculitis | Ovarian Infection | Peritonitis | Omphalitis |
| | Infectious Bronchitis | | Hemorrhagic Enteritis | |
| | | | Turkey Poult Enteritis | |
| | | | Colibacillosis | |
| | | | Coligranuloma in liver | |
| | | | and intestines | |
| Erysipelothrix | | | | Spleenitis |
| | | | | Endocarditis |
| | | | | Arthritis |
| Fusobacterium | Avian Diphtheria secondary | | | |
| | to Fowl Pox | | | |
| Klebsiella | | | | |
| Pasturella | Fowl Plague | | Fowl Cholera | Fibrinous |
| | Pasteurellosis | | | Polyserositis |
| Peptostreptococcus | | | | |
| Proteus | | | | |
| Pseudomonas | | | | |
| Rhodococcus | | | | |
| Salmonella | | | Pullorum Disease | Fowl |
| | | | White Diarrhea | Typhoid |
| | | | | Paratyphoid |
| Serratia | | | | Septicemia |
| Staphlococci | | | | Bumble-Foot |
| | | | | Arthritis |
| | | | | Breast Blister |
| Streptococci | | | | Septisemia |
| | | | | Endocarditis |
| Vibrio | | | Cholera-like Enteric Disease | |
| Haemophilus | Infectious Coryza | | | |
| Arcanobacterium | | | | |

Table 14 is a partial listing of known neuraminidase dependent bacteria and the infectious diseases associated with them in chickens, turkeys, ducks.

TABLE 15
Neuraminidase Dependent Bacteria and Other Species' Diseases
Sheep, Goats and Rabbits

| Bacteria spp: | Respiratory | Urogenital | Gastrointestinal | Other |
|---|---|---|---|---|
| Actinomyces | | | | |
| Aeromonas | | | | |
| Bacteroides | | | | Contagious Foot Rot |
| | | | Enterotoxic Diarrhea | |
| Bordetella | “Snuffles” in Rabbits | | | |
| | Bronchopneumonia | | | Septicemia |
| Brucella | | Abortion | | |
| | | Epididymitis | | |
| Campylobacter | | Abortion | | |
| | | Ovine Genital | | |
| | | Campylobacteriosis | | |
| Clostridium | | Pulpy Kidney Disease | Enterotoxemia | Tetanus |
| | | Gangrenous Mastitis | Mucoid Enteritis/Rabbits | Botulism |
| | | | | Maligant Edema |
| | | | Braxy | Big Head |
| | | | | Hemorrhagic Enterotoxemia |
| | | | | Struck |
| Corynebacterium | | | | Caseous Lymphadenitis |
| Enterobacter | | | | |
| E. coli | | Mastitis | Colibacillosis | |
| | | | Colisepticemia | |
| | | | “Watery Mouth” in Nenatal | |
| | | | Lambs | |
| Erysipelothrix | | | | Septicemia |
| | | | | Arthritis |
| | | | | Endocarditis |
| Fusobacterium | | | | Foot Abscess |
| | | | | Necrobacillosis of lips and |
| | | | | mouth |
| Klebsiella | | | | |
| Pasturella | Pleuropneumonia | Mastitis | | Septicemia |
| Peptostreptococcus | | | | |
| Proteus | | | Diarrhea in Goats. Lambs | |
| Pseudomonas | | | | Arthritis |
| | | | | Lymphangitis |
| Rhodococcus | | | | |
| Salmonella | | Abortion in Ewes | Enteritis | Septicemia |
| Serratia | | | | |
| Shigella | | | | |
| Staphlococci | | Mastitis | | Dermatitis |
| | | | | Abscess |
| | | | | Periorbital Eczema |
| | | | | Conjunctivitis |
| Streptococci | Pneumonia | Chronic Mastitis | | Arthritis |
| Haemophilus | | | | |
| Arcanobacterium | | Mastitis | | Foot Abscess |

Table 15 is a partial listing of known neuraminidase dependent bacteria and the infectious diseases associated in sheep, goats, rabbits.

TABLE 16
Clinical Trial: Tamiflu and E. coli

| Veterinarian or Clinic: | | Cat Health Clinic | | Phone: | ( 910 ) 295-2287 | |
|---|---|---|---|---|---|---|
| | Address: | 2212 Midland Road | Pinehurst | | NC | 28374 |
| | | Street | City | | State | Zip |

| | | |
|---|---|---|
| Patient: | Owner: | Vince and Peggy Meads |

| | | | | |
|---|---|---|---|---|
| Name: | Pinga | Age: | Oct. 19, 1998 | Breed: Siamese |
| | | Sex: | FS | |

Medical History:

Presented Nov. 22, 2004 for vomiting beginning on Nov. 19, 2004. Blood for CBC/Chem Profile submitted along with
urine for culture sensitivity. Started Zeniquin at 12.5 mg/day dissolved in Rebound electrolyte solution. Reglan was
given for nausea.
When seen on Nov. 24, 2004, was presented on a blanket in lateral recumbancy. Had urinated blood tinged urine
on bedding. Lab reported isolating E. coli, sensitivity pending. Other abnormal values: BUN (55 mg/dl), Phos (10.6
mg/dl), Sodium (162 mEq/L), Osm (340 mOs/L and WBC elevated at 19,100. Since Pinga had gotten progressively
worse over the course of antibiotic therapy, and now appeared to be approaching endotoxic shock Tamiflu was
begun at 2 PM. E. coli is a neuraminidase dependent bacteria.

| | | | | | | |
|---|---|---|---|---|---|---|
| Physical Exam: | Temp: | 99.9 F. | Pulse: | 140/min | Weight: | 8.06 lbs. |
| | Resp: | | % Dehy: | Slight | Parvo Test: | Not Done |

Pinga was presented laying on her side unable to sit or stand.
She had a decreased capillary refilling time and temperature was subnormal.

Tamiflu dose: 1 mg/lb . . . that dose given every 12 hours for a total of 10 treatments

| Treatment | Drugs/Fluids | Observations |
|---|---|---|
| 1st. | Dissolved 12.5 mg Zeiniquin in 12 cc of | Can not sit or stand, urinated in bed this |
| Date: | Rebound and gave PO | morning . . . urine was blood tinged. E.coli |
| Nov. 24, 2004 | Gave 12 mg Tamiflu (1 cc)/PO at 2:00 PM | cultured . . . sensitivity pending. |
| Temp: | | |
| 99.8 F. | | |
| 2nd. | Gave 12 mg Tamiflu (1 cc) at 5:35 PM | Pinga is more alert and has not vomited |
| Date: | | since receiving Tamiflu. Can not stand, |
| Nov. 24, 2004 | | but can sit upright. |
| 3rd. | Dissolved 12.5 mg Zeniquin in 12 cc of | No vomiting since starting Tamiflu..is |
| Date: | Rebound and gave PO | drinking water . . . walked 20 feet and urinated |
| Nov. 25,2004 | Gave 12 mg Tamiflu/PO at 10:15 AM | a clear yellow colored urine next to litter box. |
| Temp: | | About 1:30 AM, left bed, walked to owner's |
| 99.5 F. | | bed, jumped up and began to purr |
| 4th. | Tamiflu and Parvo Clinical Trial | Urinated, was normal yellow color . . . has |
| Date: | Gave 12 mg Tamiflu/PO at 5:30 PM | begun to walk around house . . . jumped |
| Nov. 25, 2004 | | and ran when attempted to brush . . . Pinga |
| Temp: | | is more alert in clinic |
| 98.9 F. | | |
| 5th. | Dissolved 12.5 mg Zeniquin in 15 cc | Pinga is walking around house . . . slept in |
| Date: | Rebound and gave PO at 10:30 AM | owner's bed..refused being given Rebound |
| Nov. 26, 2004 | Gave 12 mg Tamiflu/PO | by syringe . . . comes when called . . . Physical |
| Temp: | | exam is normal |
| 98.8 F. | | |
| 6th. | Gave 12 mg Tamiflu/PO at 5 PM | Urinated normal urine . . . passed 3 small |
| Date: | | firm BMs . . . jumped up to help iron clothes, |
| Nov. 26, 2004 | | vocal . . . First time temperature has been |
| Temp: | | above 100 F. |
| 100.6 F. | | |
| 7th. | Dissolved 12.5 mg Zeniquin in 15 cc | Beginning 3rd day of Tamiflu . . . Pinga is |
| Date: | Rebound | more alert . . . began to eat Wellness dry |
| Nov. 27, 2004 | Gave 12 mg Tamiflu/PO at 10:30 AM | cat food . . . had BM in litter box . . . continues |
| Temp: | | to be given Rebound via syringe at home |
| 98.8 F. | | |
| 8th. | Gave 12 mg Tamiflu/PO at 5 PM | Appears to be “normal”, alert, active and |
| Date: | | interested in surroundings . . . Dispensed |
| Nov. 27, 2004 | | CNM-EN as a semi-soft food to try at home |
| Temp: | | |
| Not Taken | | |
| 9th. | Dissolved 12.5 mg Zeniquin in 15 cc | Ate CMN-EN last night, urinated normally, |
| Date: | Rebound | almost “normal” . . . maybe weak in rear |
| Nov. 28, 2004 | Gave 12 mg Tamiflu/PO at 10:30 AM | when playing with ball . . . shows interest |
| Temp: | | when Jerry is tying shoe strings . . . this is |
| 100.1 F. | | a normal activity for Pinga |
| 10th. | Gave 12 mg Tamiflu/PO at 5:30 PM | Pinga appears to be normal . . . this is his |
| Date: | | last treatment with Tamiflu. |
| Temp: | | |
| Not Taken | | |

In Table 16, E. coli, a neuraminidase dependent bacteria, was cultured from Pinga's urine followingan acute onset of vomiting and hematuria. She failed to respond to Zeniquin, but had a dramatic reversal when TAMIFLU was started on Nov. 24, 2005 when she presented in an endotoxic condition. This case demonstrates the success of TAMIFLU in cases of E. coli enterotoxemia.

Oral Nasal Candida famata in an Adult Male Cat

An adult neutered male domestic shorthair white cat was seen at the Cat Health Clinic for a second opinion. During the past 2 weeks, the cat had been seen and treated by three other veterinary clinics and one emergency veterinary clinic for an upper respiratory infection. Typically, most feline upper respiratory infections are caused by a combination of a virus and one or more bacteria. The lesions seen in these infections are usually ulcerative and painful. This cat's presentation was different in that there were no ulcers with the primary complaint was the excessive mucous being produced resulting in a reduced ability to get air into the lungs. This excessive mucous also prevent any intake of food, leading to weight loss poor tissue oxygenation. This mucous was very viscous and tenacious leading the cat to collapse from lack of oxygen and had been kept in an oxygen cage prior to entry. Each veterinarian had placed an esophageal-gastric feeding tube only to have it fall out due to the slickness of the mucous.

Physical examination found the cat to be emaciated (5.1 lbs) who was anemic (PCV 20%), elevated liver enzymes and total bilirubin. Both FeLV/FIV tests were negative and stress induced hypothyroidism. In-house cytology mixed rod and cocci bacteria and degenerate neutrophils in the oral cavity. The most significant finding was the presence of long non-septed pseudohyphae and blastospores typical of Candida spp in samples taken from the nares, oral cavity and anterior esophagus. (photo of blastospores). There had been no response to either Doxycycline or Zithromax given during the previous 2 weeks. Typically, it is known to those skilled in the art that the use of antibiotics can have enhanced the growth of the yeast.

A tentative diagnosis of Candida spp. Infection was offered. Since Candida spp use neuraminidase as a virulence factor, oseltamivir phosphate was started at 2 mg/lb given every 12 hours. Oral Zeniquin/Molasses suspension was started to treat the mixed bacterial flora present on the cytology slides. Daily nebulization to reduce the viscosity of the exudate and Marinol was given to stimulate his appetite.

There was a dramatic decrease in the production of the oral-nasal discharge 12 hours after therapy was initiated. The patient began to eat canned cat food after 12 hrs of treatment. Within 48 hrs of treatment, the patient began to clean himself.

Itraconazole was continued for 3 weeks and the Zeniquin/Molasses was stopped after 14 days. The oseltamivir phosphate was given for 3 weeks even though the clinical signs had been resolved after 72 hours of treatment.

This case is an example of using the Theory of Biological Intervention in a clinical yeast/fungal infection. The Theory of Biological Intervention states that one can treat most infections by suppressing one of more of the pathogen's virulence factors. In this case, neuraminidase is a virulence factor of Candida famata.

Tritrichomonas Foetus in a Cat Treated with Oseltamivir Phosphate

History: Calorie was a six-year-old red tabby neutered domestic shorthair cat when he was first presented to a veterinarian for diarrhea. Both direct and flotation exams for fecal parasites were negative and he was dismissed with the gastrointestinal antibiotic Flagyl. Five weeks later, Calorie was seen by a second veterinarian where he underwent endoscopy. These studies failed to find any pathology and he was dismissed with a special GI diet and an oral antibiotic. A fecal sample was submitted to the North Carolina State College Of Veterinary Medicine (NCSCVM) to be tested for Tritrichomonas foetus.

Two days later, Calorie was seen at the Cat Health Clinic for a second opinion. A tentative diagnosis of Inflammatory Bowel Disease was offered. Treatment consisted of a SQ injection of 4 mg Azium/5 mg Vetalog, a SQ inj of Vitamin B-12 and home with oral Zithromax to treat possible Bartonella spp. Calorie returned six days later with bloody diarrhea, and a positive test for Tritrichomonas foetus from NCSCVM.

Trichomonads are spindle to pear-shaped, highly motile protozoan that divide by binary fission and are transmitted directly via the fecal-oral route. Cats that are infected with T. foetus are generally young and presented for a waxing and waning large bowel diarrhea that contains fresh blood and mucus. The diarrhea is semi formed and malodorous.

Cats infected with T. foetus have failed when treated with numerous antimicrobial drugs. Despite this failure to eradicate T. foetus, the stools always improve while taking antibiotics, only to relapse once the drug is discontinued. Cats that have failed antibiotic treatments do tend to improve after a period of infection lasting over two years. Ronidazole, a nitroimidazole, has been shown to be effective in curing cats with T. foetus infections. Unfortunately, ronidazole can be potentially carcinogenic and humans should avoid exposure to this drug. In cats given ronidazole have developed ataxia, nystagmus and behavior changes. These signs of neurotoxicity are reversible when ronidazole is discontinued. Given the choice of their cat developing signs of neurotoxicity and a good chance of a spontaneous resolution of T. foetus in two years, most clients choose not to treat with ronidazole.

Oseltamivir phosphate was given at the dose of 2 mg/lb every 12 hours for a total of 10 treatments. Photographs were taken of every stool during the treatment. The stool went from a bloody gravy (Bristol 6) to a non-bloody formed stool (Bristol 4) after the 5th treatment. The treatments were completed and there has been no relapse in over four years.

Calorie is a typical case of feline T. foetus with relapsing bloody diarrhea over a period of six months. Once the diagnosis of T. foetus was made, the decision was made to not try and kill the T. foetus organism with ronidazole, but to intervene biologically by suppressing T. foetus' ability to secrete neuraminidase and thus prevent it from harvesting neuramic molecules from the host. By suppressing one of T. foetus' virulence factors, we were able to stop the infection. Suppressing T. foetus' ability to secrete neuraminidase proved to be a safe economical treatment for a clinical disease.

### Resistant Bacteria

In yet another example illustrating the use of oseltamivir phosphate to treat a bacterial infections resistant to antibiotics, such as Enterococcus faccalis, in which the pathogen is a neuraminidase producer, specifically where the most of the potential drugs were ineffective (bacteria was resistant). In addition, bacterial sinusitis infections in the frontal sinuses can be almost impossible to treat.

The pathogen was a gram (+) rod bacteria that produces neuraminidase as a virulence factor. By using a neuraminidase inhibitor, for example, oseltamivir phosphate to suppress bacterial salidase, we were able to totally resolve this problem by administering 1 mg/ml, q. 12 h of oseltamivir phosphate for ten consecutive treatments.

A shelter cat with a bulging area in the area of the frontal sinuses due to pus from a very resistant bacteria. Frontal sinus infections typically can be extremely difficult to treat due to the location (a cavity in the skull surrounded by bone). Most of these cases require surgical removal of the nasal bone followed inserting tubes that allow the area to be flushed daily for weeks usually with little progress.

Oseltamivir phosphate was given to the cat, in addition to an antibiotic, that changed the course of the disease.

Necrotic Enteritis (Clostridium perfringens) in Poultry Treated with Tamerindus indicus and Combretum fragrans

### Materials and Methods

A. Experimental Ration An unmedicated commercial type chicken started compounded with feedstuffs commonly used in the United States was formulated. This ration (in mash forms) was fed ad libitum from the date of chick arrival until Day 28 of the study. The diet formulation was included in the source data. Experimental treatment feeds were prepare from this basal starter feed. Quantities of all basal feed and test articles used to prepare treatment batches were documented. Treatment feeds were mixed at SPR to assure a uniform distribution of respective test article. The feed was transferred to Building #2 and distributed among cages of the same treatment.
Feed samples: One each from the beginning, middle and end of each batch of treatment diet were collected an mixed to form a composite sample. One composite sample was taken from the composite for each treatment and sent to Jack J. Broadhurst, DVM.
B. Animals: On Jul. 18, 2013, day of hatch male broiler chicks were obtained from Cobb-Vantress, Cleveland, Ga. The strain was Cobb X Cobb 500. Breeder flock information was recorded. At the hatchery, the birds were sexed and received routine vaccinations. Only healthy appearing chicks were used in the study. Number and disposition of all birds not used for allocation were documented.
C. Housing: Upon arrival, chicks were raised in Petersime battery cages. At placement the birds were fed the treatment feeds. The Petersime batteries were located in Building #2 at SPR, an insulated, concrete floored, metal structure that measures 40 ft by 100 ft in a north-south direction. The floor space per animal was 0.63 sq.ft/bird. The feeder space per bird was 8 birds/24×3.5 inch feeder. Thermostatically controlled gas furnace/air conditioner maintained uniform temperature. Even illumination was provided.

### Procedures

A. Bird Allocation and Cage Randomization: Assignment of treatments to cages was performed by SPR. Cages were blocked by location in the battery with block size equal to treatments (6 cages per block). The study began when the birds were placed (day of hatch) (DOT 0) at which time they were allocated to the experimental cages. Only healthy birds were selected. On DOT 0, group body weights were recorded by cage. No birds were replaced during the course of stay.
B. Disease Induction: Feed and water were available ad libitum throughout the trial. On DOT 14, all birds were orally inoculated with coccidial inoculum containing approximately 5,000 oocysts of E. maxima per bird. Coccidial oocyst inoculation procedures are described in SPR SOP. Starting on DOT 19, all birds, except Treatment 1, were given a broth culture of C. perfringens 10⁸ cfu/ml. The birds were administered a fresh broth culture once daily for 3 days (on DOTs 19, 20 and 21).
C. DOT 0, 14, 21 and 28 Weights: All birds weighed by cage on DOT 0, 14, 21 and 28. Feed was weighed in on DOT 0 and remaining feed was weighed on DOT 14, 21 and 28. The trial was terminated on DOT 28.
D. Necrotic Enteritis Intestinal Lesion Scoring: On DOT 21, three birds from each cage were selected, sacrificed, weighed and examined for the degree of presence of Necrotic Enteritis lesion. The scoring was based on a 0 to 3 score, with 0 being normal and 3 being the most sever.

### E. Management:

1. The facility was checked thoroughly to assure that all cages had water and that feed was available in every cage. The building temperature's range was maintained at an appropriate temperature for the age of the birds.

2. Even, continuous illumination was provided by fluorescent lamps hung vertically along the wall.

3. Feed and water were given ad libitum.

4. In accordance with SPR's standard operating procedures (SOPs), the cages were checked twice daily. Observations included were the availability of feed and water, temperature control, and any unusual conditions. The birds were watched closely for any abnormal reactions.

5. When mortality birds were removed from cages, the cage number, date, weight of the bird, sex and probably cause of death were recorded.

DATA ANALYSIS: Means for cage, weight gain, feed consumption, feed conversion, lesion scores, and mortality were calculated.
DISPOSAL of ANIMALS and Feed: All birds were buried in SPR's pit as described in SPR SOPs. Records of disposition were included in the source data.

TABLE 17
Comparative Efficacy of a 50/50 combination of Tamerindus indicus and Combretum
fragrans to AGP administered in the feed for the Control of Necrotic Enteritis caused by
Clostridium perfringens in Broiler Chickens

| | Feed Consumption | | Feed Conversion | | Weight Gain | |
|---|---|---|---|---|---|---|
| Treatment | Day 0-21 | Day 14-21 | Day 0-21 | Day 14-21 | Day 0-21 | Day 14-21 |
| 1) Nonmed, Noninfect | 6.694 bc | 3.187 c | 1.764 d | 1.827 c | 0.482 a | 0.226 a |
| 2) Nonmed, Infected | 7.080 ab | 3.513 ab | 2.157 a | 2.713 a | 0.418 b | 0.174 d |
| 3) Virginamyein 20 g/t | 7.151 a | 3.468 a | 1.894 c | 2.141 b | 0.480 a | 0.213 ab |
| 4) Tamerindus (0.25%) & | 6.915 abc | 3.342 abc | 2.044 ab | 2.381 b | 0.435 b | 0.188 bcd |
| Combretum (0.25%) | | | | | | |
| 5) Tamerindus (0.50%) & | 6.479 c | 3.153 c | 2.020 bc | 2.191 b | 0.404 b | 0.183 cd |
| Combretum (0.50%) | | | | | | |
| 6) Tamerindus (1.0%) & | 6.740 bc | 3.279 bc | 1.998 bc | 2.129 ab | 0.432 b | 0.203 abc |
| Combretum (1.0%) | | | | | | |
| | Feed Consumption | | Feed Conversion | | Weight Gain | |
| Treatment | Day 0-28 | Day 14-28 | Day 0-28 | Day 14-28 | Day 0-28 | Day 14-28 |
| 1) Nonmed, Noninfect | 8.915 ab | 5.408 a | 1.818 c | 1.902 b | 0.746 a | 0.490 a |
| 2) Nonmed, Infected | 8.802 ab | 5.235 a | 2.140 a | 2.458 a | 0.651 c | 0.406 c |
| 3) Virginamyein 20 g/t | 9.406 a | 5.723 a | 1.878 bc | 1.999 b | 0.747 a | 0.480 ab |
| 4) Tamerindus (0.25%) & | 8.543 ab | 4.970 a | 1.913 bc | 2.016 b | 0.702 ab | 0.454 ab |
| Combretum (0.25%) | | | | | | |
| 5) Tamerindus (0.50%) & | 8.295 b | 4.969 a | 1.954 b | 2.015 b | 0.630 c | 0.409 c |
| Combretum (0.50%) | | | | | | |
| 6) Tamerindus (1.0%) & | 8.467 b | 5.006 a | 1.887 bc | 1.882 b | 0.672 bc | 0.444 bc |
| Combretum (1.0%) | | | | | | |

| | NE (0-3) | % NE |
|---|---|---|
| Treatment | Lesion Score | Mortality |
| 1) Nonmed, Noninfect | 0.0 b | 0.0 b |
| 2) Nonmed, Infected | 0.4 a | 14.1 a |
| 3) Virginamvein 20 g/t | 0.2 ab | 0.0 b |
| 4) Tamerindus (0.25%) & Combretum (0.25%) | 0.3 ab | 10.9 a |
| 5) Tamerindus (0.50%) & Combretum (0.50%) | 0.3 ab | 4.7 ab |
| 6) Tamerindus (1.0%) & Combretum (1.0%) | 0.1 ab | 1.6 b |

### Anaerobic Antibiotic Resistant Nosocomial Infection

History: On Aug. 20, 2013, a forty-three years old Caucasian male living in Lyon, France underwent an arthroscopic procedure on his right knee. The patient was instructed to begin Paracetamol to control pain associated with the surgery. Paracetamol is a French OTC product that contains acetaminophen).

He returned on Sep. 13, 2013 complaining that his right knee was swollen and painful. An aerobic bacterial culture was submitted on the synovial fluid that was removed during an arthroscopic procedure that also removed an osseous spur. Staphylococcus lugdunensis was isolated from the sample submitted. Staphylococcus lugdunensis is an aerobic Gram positive cocci frequently associated with orthopedic surgery and is susceptible to multiple antibiotics. On Sep. 20, 2013, the patient was started on two oral antibiotics known to effective against Staphylococcus lugdunesis: Pyostacine (500 mg) and Oflocet (200 mg). Pyostacine (Pristinamycin) is effective against Staphylococcus and Streptococcus infections. Oflocet (Ofloxacine) is effective against Staphylococcus, Streptococcus, Proteus, Neisseria, Chlamydia, Escherichia, Klebsiella, Pseudomonas and Citrobacteria infections.

On Oct. 1, 2013, a third arthroscopic procedure was done and synovial fluid removed and submitted for culture. This second aerobic culture was reported out as “No Growth”. There had been no change in the degree of pain and swelling while taking Pyostacine and Oflocet. The patient was told to remain sedentary and continue to use a crutch if he needed to move around his apartment. Paracetamol was replaced with oral Bi-Profenid (ketoprofene, an NSAID used for pain secondary to osteoarthritis).

A recheck on Oct. 13, 2013 found that there had been no change in the degree of pain and swelling. The patient was ordered to stop Pyostacine and Oflocet. Arrangements were made for a nurse to come to the patient's apartment once a day and to administer 2 gm of Rocephine once a day by a slow drip of IV fluids. This treatment began on Oct. 18, 2013 and ended on Oct. 30, 2013.

Rocephine (Cetriaxone) has proven to be effective against: Gram negative bacteria: Acinetobacter, Enterobacter, Escherichia, Klebsilella, Moraxella, Neisseria, Proteus, Pseudomonas and Serratia spp.

Gram positive bacteria: Staphylococcus and Streptococcus spp.
Anaerobic bacteria: Bacteroides, Clostridium and Peptostreptococcus spp.

No antibiotics were given after the last Rocephine infusion on Oct. 30, 2013. In an attempt to better control the patient's pain, Bi-Profenid was replaced with Lamoline (combination of acetaminophen+opiate+caffeine) and Tramadol which is a mild narcotic used for moderate to sever pain.

On Nov. 17, 2013, a recheck examination found that there had been no change in the degree of pain and swelling. A third aerobic culture was submitted using synovial fluid removed from the knee joint. This was reported out as a “No Growth”. The patient was ordered to continue to take Lamoline and Tramadol to control pain and to limit activity to his apartment and use crutches when required to walk. The doctors discussed amputation as the last resort to remove the source of pain and swelling.

On Nov. 26, 2013, the patient contacted Dr. Jack Broadhurst and asked if there were any alternatives to amputation. After reviewing his medical history, Dr. Broadhurst suggested that the French doctors had not ruled out an anaerobic, antibiotic resistant nosocomial (hospital source) infection.

In the United States, Enterococcus faecalis is one of the leading causes of nosocomial infection and in many cases has been reported to resistant to all available antibiotics. Dr. Broadhurst has had success treating E. faecalis with a neuraminidase inhibitor (Oseltamivir Phosphate). He suggested that the patient locate a source of Oseltamivir Phosphate and start taking 1 mg/lb (0.5 mg/kg) every 12 hours. If there was no change in the degree of swelling or pain, after two doses, the patient should increase the dose for the remaining doses to 2 mg/lb (1 mg/kg).

On Nov. 29, 2013, the patient obtained Oseltamivir Phosphate and began taking 150 mg every 12 hours.

| Date | AM/PM | Clinical Response |
|---|---|---|
| Nov. 29, 2013 | PM | Initial dose of 150 mg (PO) |
| Nov. 30, 2013 | AM/PM | All pain gone, swelling |
| | | reduced 30% |
| Dec. 1, 2013 | AM/PM | Got out of bed, able to stand |
| | | without crutches, walked |
| Dec. 2, 2013 | AM/PM | Walked down stairs, walk |
| | | to local park, kicked soccer |
| | | ball |
| Dec. 3, 2013 | AM/PM | Biked to park, normal gait |
| | | played goalie in soccer |
| | | game |
| Dec. 4-5, 2013 | AM/PM | Normal gait and activity |
| Dec. 6-13, 2013 | AM | Reduced to 150 mg per day |
| Dec. 14, 2013-Current Date | | No relapse |

The foregoing is merely illustrative of the invention and is not intended to limit the invention to the disclosed compounds and the particular embodiments. Variations and changes which are obvious to one skilled in the art are intended to be within the scope and nature of the invention. Thus, the true scope of the present invention is not limited to any one of the foregoing exemplary embodiments.

What is claimed is:

1. A method for inhibiting neuraminidase dependent bacterial infections that are not viral generated dependent infections, wherein the neuraminidase dependent bacterial infections that are not viral generated infections are from a disease-causing microorganism dependent on sialic acid metabolism, whereby the microorganism comprises a bacterial species selected from the group consisting of Clostridium, Enterococcus, Enterobacteriaceae, Escherichia coli, Acinetobacter, Pasteurella, Campylobacterium, Klebsiella, Pseudomonas, Neisseria, Salmonella, Shigella, Staphylococcus, Streptococcus and Vibrio, comprising administering to an animal in need thereof a therapeutically effective amount of a composition comprising one or more compounds, wherein one of the compounds comprises oseltamivir phosphate.

2. A method for treating neuraminidase dependent bacterial infections from a disease-causing microorganism dependent on sialic acid metabolism, whereby the microorganism comprises a bacterial species selected from the group consisting of Clostridium, Enterococcus, Enterobacteriaceae, Escherichia coli, Acinetobacter, Pasteurella, Campylobacterium, Klebsiella, Pseudomonas, Neisseria, Salmonella, Shigella, Staphylococcus, Streptococcus and Vibrio, comprising administering to an animal in need thereof a therapeutically effective amount of a composition comprising one or more compounds, wherein one of the compounds comprises oseltamivir phosphate.

3. The method of claim 1, wherein the therapeutically effective amount of oseltamivir phosphate is from about 0.6 mg/lb to about 12 mg/lb.

4. The method of claim 3, wherein the therapeutically effective amount of oseltamivir phosphate is from about 0.3 mg/lb to about 10 mg/lb.

5. The method of claim 2, wherein the therapeutically effective amount of oseltamivir phosphate is from about 0.6 mg/lb to about 12 mg/lb.

6. The method of claim 5, wherein the therapeutically effective amount of oseltamivir phosphate is from about 0.3 mg/lb to about 10 mg/lb.
